(19) United States
(12) Patent Application Publication
(10) Pub. No.: US 2007/0099970 A1
(43) Pub. Date: May 3, 2007

(54) Immunomodulatory compounds that target and inhibit the pY'binding site of tyrosene kinase p56 LCK SH2 domain

(76) Inventors: Alexander Mackerell, Baltimore, MD (US); Jun Hayashi, Ellicott City, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/507,038

(22) Filed: Aug. 21, 2006

Related U.S. Application Data
(60) Provisional application No. 60/709,972, filed on Aug. 19, 2005.

(51) Int. Cl. A61K 31/427; A61K 31/426

(52) U.S. Cl. 514/369

(57) ABSTRACT

Small molecular-weight non-peptidic compounds block Lck SH2 domain-dependent interactions. The inhibitors omit phosphotyrosine (pY) or related moieties.

### PRIORITY

This application claims priority to U.S. provisional application 60/709,972, filed on Aug. 19, 2005.

### STATEMENT AS TO RIGHTS TO INVENTIONS MADE UNDER FEDERALLY SPONSORED RESEARCH AND DEVELOPMENT

The invention was made with United States Government support under Contract No. CA095200 from the National Institutes of Health. The United States Government has certain rights in the invention.

The protein p56 Lck (Lymphoid T cell tyrosine kinase) is a member of the Src family of tyrosine kinases and is predominantly expressed in T lymphocytes and natural killer cells where it plays a critical role in T-cell-mediated immune responses.^{1,2} p56 Lck is responsible for the phosphorylation of conserved tyrosine residues of CD3 chains, called immunoreceptor tyrosine-based activation motifs (ITAMs), the first step required for T cell activation signaling cascades.^{3,4} Failure of the p56 Lck SH2 domain to bind to ITAMs of CD3 will hamper the T cell receptor (TCR) proximal activation process and suppress the downstream T cell activation signaling cascades.^{3,5} Lck participates in phosphotyrosine (pY)-dependent protein-protein interactions through its modular binding units, called src Homology-2 (SH2) domains.⁶ Accordingly, ligands that are able to block Lck SH2 domain-dependent protein-protein interactions will ultimately find therapeutic utility as immunosuppressants and in the treatment of T cell leukemias, lymphomas and autoimmune diseases such as rheumatoid arthritis.^{2,7}

A phosphopeptide library screen has identified a preferred pY containing peptide binding sequence Ac-pY-E-E-I for the Lck SH2 domain.⁸ This tetrapeptide is an attractive lead structure for the rational design of agents to compete with the SH2 domain's natural ligands. Unfortunately, the tetrapeptide Ac-pY-E-E-I has several undesirable features that hinder its ability to elicit a response in cell-based assays of T-cell activation. First, the phosphate group, an essential element for peptide binding to the SH2 domain, is metabolically unstable to phosphatases present in cells and, secondly, the five negative charges at physiological pH and the high peptidic character may limit its ability to reach efficacious concentrations inside the cell. Due to the conservation of the pY binding site, a pY or similar functional group is strictly required to maintain the peptide binding.¹ Attempts to design SH2 inhibitors with high receptor binding affinity, chemical stability and minimally charged phosphate group replacements have met with limited success.⁹⁻¹¹ Accordingly, novel approaches towards the identification of p56 Lck SH2 domain inhibitors that avoid the problems associated with the strategies applied to date are required.

High resolution X-ray structures of the Lck SH2 domain complexed with the pY-E-E-I type peptide have provided a 3D molecular map revealing that the pY and Ile residues of the peptide are bound to two well-defined cavities, referred to as the pY and pY+3 binding sites, where the interaction resembles a two-pronged plug engaging a two-holed socket.¹ This binding mode is consistent with experimental observation that SH2 affinity is strongly dependent on the pY and Ile side chains.¹² Moreover, site mutations of amino acid residues in pY+3 binding site switched the binding specificity¹³⁻¹⁵, which has led to the proposal that the pY+3 binding pocket is also important for specific binding.¹² Thus, the pY+3 site represents a novel target site for the application of rational drug design approaches to identify non-peptidic, specific inhibitors of the p56 Lck SH2 domain.

By using virtual screening methods one can provide an indication as to whether an inventive compound has the proper “fit” to, and is complementary to, a region of the protein which is important for specificity of binding, e.g., a p56^{lck} SH2 domain, as opposed to, e.g., Hck, Fyn, Src, Shc or ZAP-70 SH2 domains. In particular, such methods can indicate whether a compound is complementary to the pY+3 binding site of p56^{lck}. The terms “specific binding” or “specificity of binding” as used herein mean that an inventive compound interacts with, or forms or undergoes a physical association with, a particular SH2 domain (e.g., a p56^{lck} SH2 domain) with a higher affinity, e.g., a higher degree of selectivity, than for other protein moieties (e.g., SH2 domains of other protein kinases).

Virtual screening techniques followed by experimental assays have been used to identify small molecular-weight (MW) non-peptidic compounds targeting the pY+3 binding site that are potent inhibitors of the Lck SH2 domain.

In one embodiment, the invention relates to a method of achieving an immunomodulatory effect in a patient in need thereof, comprising administering an effective amount of one or more of the compounds 276-1 to 276-29, 99-1 to 99-37, 73-1 to 73-33, 92-1 to 92-21, 103-1 to 103-20, 146-1 to 146-22, 245-1 to 245-26, 139-1 to 139-26, 149-1 to 149-30, 275-1 to 275-23, 162-1 to 162-30, 262-1 to 262-22 and a compound of formula I, or a salt thereof, hereinafter collectively referred to as “compounds of the invention.” Compounds of formula I are described next and the rest of the compounds can be found in tables 1 through 12.

Compounds of formula I are 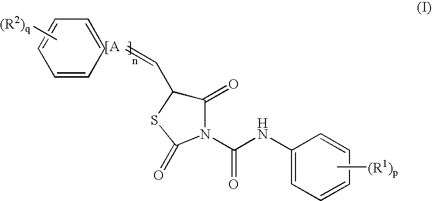
wherein
A is a 5-membered aromatic ring in which optionally a carbon is replaced by a nitrogen or oxygen, and which optionally is substituted in 0, 1 or 2 places with a C₁₋₄ alkyl group, or is a straight chain or branched C₁₋₄ alkenylene group,
n is 0 or 1,
p is 0, 1 or 2,
q is 0, 1 or 2, and
R¹ and R² are, each independently, a halogen atom, a carboxylic acid group, a hydroxyl group, a —C(O)O—C₁₋₄ alkyl group, or a C₁₋₆ alkyl group that is optionally substituted with a hydroxyl group or with a carboxylic acid group,
   wherein, preferably,
R¹ is a C₁₋₄ alkyl group, a halogen atom, or a carboxylic acid group, and
R² is a halogen atom, a carboxylic acid group, a hydroxyl group, a —C(O)O—C₁₋₄ alkyl group, or a C₁₋₄ alkyl group that is optionally substituted with a hydroxyl group.

Preference is given to compounds of formula XVIII, wherein
A is 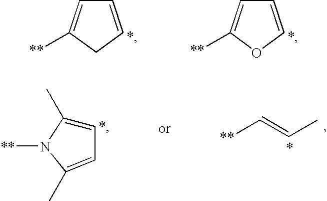
   wherein the * denotes the bonding location to the alkenylene group of the compound of formula I, and ** denotes the bonding location to the phenyl ring of the compound of formula I that is adjacent to the group A,
R¹ is CH₃, F, or COOH,
R² is CH₃, Cl, COOH, C(O)OCH₃, OH, or CH₂OH,
n is 0 or 1,
p is0or 1, and
q is 0, 1 or 2.

Further preference is given to compounds wherein an R² in the compounds of formula XVIII is a meta or para position acid group, e.g., hydroxyl group or carboxylic acid group, preferably a carboxylic acid group.

Further preference is given to compounds of formula XVIII wherein the group A is

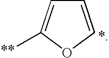

More preferred are compounds of formula XVIII which have both a R² as an acid group, e.g., hydroxyl group or carboxylic acid group, preferably a carboxylic acid group, in a meta or para position and have the group A as

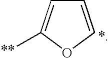

Preferred in the above embodiment, and also in other embodiments herein, are compounds 276-1 to 276-29, 99-1 to 99-37, 73-1 to 73-33, 92-1 to 92-21, 103-1 to 103-20, 146-1 to 146-22, 245-1 to 245-26, and compounds of formula I and more preferred are compounds 276-1 to 276-29, 99-1 to 99-37, 73-1 to 73-33, and 92-1 to 92-21.

More preferred are compounds with higher inhibition values shown in FIGS. 1-4. Preferred are compounds, for example, having experimental inhibition values above 20%, for example, above 25%, and above 40%. Even more preferred are compounds with experimental inhibition values above 60%, and even more so compounds with values above 80%.

In another embodiment, the invention relates to a method for achieving an antineoplastic effect in a patient in need thereof, comprising administering an effective amount of a compound of the invention or a salt thereof.

In another embodiment, the invention relates to a method of modulating the binding of a p56^{lck} molecule via an SH2 domain thereof to a corresponding cellular binding protein, and/or modulating the activity of a p56^{lck} molecule via binding to an SH2 domain thereof, comprising binding to an SH2 domain of said p56^{lck} molecule to a compound of the invention or a salt thereof.

In another embodiment, the invention relates to a method of inhibiting hyperproliferative cell growth in a patient in need thereof, comprising administering an effective amount of a compound of the invention or a salt thereof.

In further embodiments according to the invention, the compounds of the invention are effective in affecting immunosuppression in a patient.

In further aspects, the compounds of the invention are useful in treating patients with an autoimmune disease or patients who suffer from a depressed immune system.

In a preferred embodiment, the compounds of the invention are used to treat a patient who suffers from a transplant rejection.

In another preferred embodiment, the compounds of the invention a treat rheumatoid arthritis.

In further aspects, the compounds of the invention are used to treat a patient with a neoplasm or a hyperplasia, or a patient who has a benign or malignant tumor, or a patient who suffers from leukemia, lymphoma, ovarian cancer or breast cancer.

In a further aspect, the invention relates to a method of achieving an immunomodulatory effect, achieving an antineoplastic effect, or inhibiting hyperproliferative cell growth in a patient in need thereof, comprising administering to said patient an effective amount of a compound that hydrogen bonds to residues Lys179, Lys182, and Arg184 of the Lck SH2 domain of a p56^{lck} molecule.

In yet a further aspect, the invention relates to a method of achieving an immunomodulatory effect, achieving an antineoplastic effect, or inhibiting hyperproliferative cell growth in a patient in need thereof, comprising administering to said patient an effective amount of a compound that hydrogen bonds to residues Lys179, Lys182, and Arg184 of the Lck SH2 domain of a p56^{lck} molecule, wherein the compounds of formulae I and VI of U.S. application Ser. No. 10/582,640 are excluded. U.S. application Ser. No. 10/582,640 is incorporated by reference herein.

All compounds of the invention can be prepared fully conventionally, using known reaction chemistry, starting from known materials or materials conventionally preparable. [See, e.g., Houben-Weyl, Methoden der Organischen Chemie [Methods of Organic Chemistry], Georg-Thieme-Verlag, Stuttgart]. Most compounds of the invention are readily available from standard sources, such as chemical supply houses, or can be generated from commercially available compounds by routine modifications. All tested compounds were purchased from commercial vendors, e.g., Chembridge whose website is http://www.chembridge.com; Chemdiv whose website is http://www.chemdiv.com; Maybridge whose website is http://www.maybridge.com; Mdd whose website is http://www.worldmolecules.com; Nanosyn whose website is www.nanosyn.com; Specs whose website is http://www.specs.net; Timtec (st) whose website is http://www.timtec.net; Tripos whose website is http://www.tripos.com. All compounds described in the application are known compounds.

Among the advantages of the compounds of the invention are that the molecules are not susceptible to enzymatic hydrolysis (as are certain peptide and protein modulators of protein tyrosine kinase activity), and that they exhibit good cell permeability characteristics.

Without wishing to be bound to any particular mechanism, this invention relates, e.g., to compounds that interact specifically with proteins, e.g., protein tyrosine kinases, which are involved in intracellular signaling pathways, in particular to compounds that interact with SH2 domains of such tyrosine kinases, and more particularly to compounds that interact with an SH2 domain of the p56^{lck} src family tyrosine kinase. Among other functions, the p56^{lck} protein is involved in signal transduction pathways involved in T cell antigen receptor activation signaling required for mounting an active immune response, and in aspects of cell proliferation, e.g., proliferation of neoplastic cells. It is proposed that compounds of the invention, by interacting with p56^{lck}, particularly with an SH2 domain thereof, modulate the kinase activity of the protein and/or modulate its ability to interact with a corresponding cellular binding protein, and thereby modulate immune responses, directly or indirectly, and neoplastic cell proliferation. Compounds of the invention can either enhance or inhibit signal transduction pathways, including downstream signal transduction processes in a signal transduction pathway, or they can be biphasic, either enhancing or inhibiting, depending on conditions. The effect of any given compound can be routinely determined by screening in one or more of the assays described herein or other fully conventional assays.

The non-catalytic domains of p56^{lck} kinase, e.g. the SH2 domain(s), mediate specific intramolecular and intermolecular interactions that are important for the regulation of p56^{lck} function; they exert both negative and positive effects on kinase activity. In general, the intramolecular interaction keeps p56^{lck} in an inactive state, and the intermolecular interactions facilitate p56^{lck} kinase action. For example, the SH2 domain can positively regulate p56^{lck} enzymatic activity by targeting p56^{lck} to specific cellular sites [ITAM (immunoreceptor tyrosine based activation motifs) phosphotyrosines containing peptides] where substrate phosphorylation is needed; and p56^{lck} that is bound to phosphtyrosine sites via its SH2 domain can exhibit higher enzymatic activity, thereby enhancing further phosphorylation of substrates. Without wishing to be bound to any particular mechanism as to how this is accomplished, it is proposed that the compounds which bind to the SH2 domain can either increase (activate, enhance, stimulate), decrease (suppress, inhibit, depress), or have no effect on, kinase activity and attendant cellular phosphorylation events (e.g., processes involved in intracellular signaling).

p56^{lck} plays an important role in modulating immune responses. p56^{lck} is a T-cell specific kinase, the majority of which is associated with CD4 (in T_{H} cells) and CD8 (in cytotoxic T cells). The p56^{lck} kinase is responsible, e.g., for an early step in activating T cells—the phosphorylation of ITAM in CD3 chains—which in turn initiates multiple intracellular cascades of biochemical events leading to, e.g., actin polymerization, enhanced gene transcription, cellular proliferation and differentiation. p56^{lck} also plays an important role in a second important step in the activation of T cells-immunological synapse formation. The compounds of the invention can modulate the immune response by, e.g. modulating T-cell activation, or indirectly by modulating downstream processes of a signal transduction pathway. As used in this application, the term “modulate” means to change, e.g., to increase (activate, enhance, stimulate) or decrease (suppress, inhibit, depress) a reaction or an activity. Compounds of the invention can be said to modulate the binding of a p56^{lck} SH2 domain to a “corresponding cellular binding protein,” which term, as used herein, refers to any cellular binding protein whose binding to p56^{lck} is mediated by SH2 domains. Such corresponding cellular binding proteins include, e.g., CD3 chains, ZAP-70, p62, Lad, CD45, Sam68 or the like.

Many protein tyrosine kinases play a role in regulating cellular events, including gene activation and/or regulation, and thus, e.g., in cell proliferation. p56^{lck} is a proto-oncogene, which has been implicated in a number of pathological conditions that involve undesirable hyperproliferation of cells. For example, overexpression of constitutively active p56^{lck} has been observed in murine and human lymphomas, suggesting that p56^{lck}-mediated phosphorylation of cellular proteins stimulates lymphocyte proliferation. In addition, overexpression and activation of p56^{lck} appears to play an important role in the human lymphoid cell transformation induced by Epstein-Barr virus and Herpesvirus Saimiri. Moreover, transgenic mice overexpressing wild type p56^{lck} and a constitutively active form of p56^{lck} in thymocytes develop thymoma, suggesting that even the overexpression of wild type p56^{lck} can transform cells under these conditions. Compounds of the invention, e.g. compounds which inhibit p56^{lck} activity, are useful for the treatment of conditions involving hyperproliferative cell growth, either in vitro (e.g., transformed cells) or in vivo. Conditions which can be treated or prevented by the compounds of the invention include, e.g., a variety of neoplasms, including benign or malignant tumors, a variety of hyperplasias, or the like. Compounds of the invention can achieve the inhibition and/or reversion of undesired hyperproliferative cell growth involved in such conditions.

As used herein, the term “hyperproliferative cell growth” refers to excess cell proliferation. The excess cell proliferation is relative to that occurring with the same type of cell in the general population and/or the same type of cell obtained from a patient at an earlier time. “Hyperproliferative cell disorders” refer to disorders where an excess cell proliferation of one or more subsets of cells in a multicellular organism occurs, resulting in harm (e.g., discomfort or decreased life expectancy) to the multicellular organism. The excess cell proliferation can be determined by reference to the general population and/or by reference to a particular patient (e.g., at an earlier point in the patient's life). Hyperproliferative cell disorders can occur in different types of animals and in humans, and produce different physical manifestations depending upon the affected cells. Hyperproliferative cell disorders include, e.g., cancers, blood vessel proliferative disorders, fibrotic disorders, and autoimmune disorders.

Activities and other properties of the compounds of the invention (and comparisons of those activities to those of art-recognized, comparison compounds) can be measured by any of a variety of conventional procedures.

A variety of in vitro assays can be used to measure biological and/or chemical properties of the compounds, and are conventional in the art. For example, in vitro binding studies can determine the affinity and the specificity of binding of the compounds, e.g., to a p56^{lck} SH2 domain. Assay Example 4 illustrates a method to determine K_{D} and IC₅₀ values, using tritiated compounds and purified, recombinant p56^{lck} SH2 domains. Similar assays can show that compounds bind selectively in vitro to a particular site, e.g., to the p56^{lck} SH2 domain, but not to other sites, e.g., Hck, Fyn, Src, Shc or ZAP-70 SH2 domains. Assay Example 5 illustrates an in vitro co-immunoprecipitation (IP) kinase assay. Again, similar assays can show the specificity of binding of the compounds. Assay Example 6 illustrates an assay to determine specificity of the binding.

Other conventional in vitro assays can measure the effect (e.g., inhibition or enhancement) of the compounds on biological activities associated with tyrosine protein kinases, e.g., p56^{lck}. p56^{lck} activities which are involved in immune responses include, e.g., the phosphorylation of, e.g., tyrosine in the ITAM consensus sequence present in certain molecules, e.g., CD3 chains; immunological synapse formation, e.g., with corresponding cellular binding proteins; or the like. Assay Example 1 illustrates an in vitro assay for Jurkat cell-activation-dependent phosphorylation, an activity that is correlated with T-cell activation. Assay Example 2 illustrates an in vitro assay for cell viability, which indicates if a compound is cytotoxic or cytostatic. Assay Example 3 illustrates an in vitro assay for IL-2 production, an activity which is correlated with T-cell activation. Assay Example 7 illustrates a mixed lymphocyte culture assay.

A variety of in vivo assays can be used to demonstrate immunomodulatory properties of the compounds. Such in vivo assays, and appropriate animal models for disease conditions that can be treated with the compounds, are well-known to those of skill in the art. For example, animal models for rheumatoid arthritis are illustrated in Assay Example 8.

Assays to measure the effect of compounds (e.g., phosphotyrosine kinase inhibitors) on cell growth (proliferation) and cell transformation are conventional. A variety of typical assays are described, e.g., in Kelloff, G. J., et al., Cancer Epidemiol Biomarkers Prev., 1996. 5(8), p. 657-66; Wakeling, A. E., et al., Breast Cancer Res Treat, 1996, 38(1), 67-73; Yano, S., et al., Clin Cancer Res, 2000, 6(3), p. 957-65; Reedy, K. B., et al., Cancer Res, 1992, 52(13), p.3636-41; Peterson, G. and S. Barnes, Prostate, 1993; 22(4), p. 335-45; Scholar, E. M. and M. L. Toews, Cancer Lett, 1994, 87(2); 159-62; Spinozzi, F., et al., Leuk Res, 1994, 18(6), p. 431-9; Kondapaka, B. S. and K. B. Reddy, Mol Cell Endocrinol, 1996, 117(1), p. 53-8; Moasser, M. M., et al., Cancer Res, 1999, 59(24), p. 6145-52; Li, Y., M. Bhuivan & F. H. Sarkar, Int J Oncol, 1999, 15(3), p. 525-33; Baguley, B. C., et al. Eur J Cancer, 1998, 34(7), p. 1086-90; and Bhatia, R., H. A. Munthe, and C. M. Verfaillie, Leukemia, 1998, 12(11), p. 1708-17.

Variations of the assays described herein, as well as other conventional assays, are well known in the art. Such assays can, of course, be adapted to a high throughput format, using conventional procedures.

The compounds of the invention are effective for binding to, e.g., p56^{lck} SH2 domains, and for modulating the activity of, e.g., p56^{lck} in animals, e.g., mammals, such as mouse, rat, rabbit, pets, (e.g., mammals, birds, reptiles, fish, amphibians), domestic (e.g., farm) animals, and primates, especially humans. The inventive compounds exhibit, e.g., immunomodulatory activity and/or antineoplastic activity, and are effective in treating diseases in which, e.g., aberrant regulation or activity of tyrosine kinase (e.g., p56^{lck}) and/or intracellular signaling responses are involved. For example, compounds which stimulate immune responses (immunostimulants) are useful for treating or preventing naturally occurring immunosuppression or immunosuppression from a variety of conditions and diseases. Compounds which depress immune responses (immunosuppressants) are useful for treating or preventing, e.g., autoimmune diseases which are characterized by inflammatory phenomena and destruction of tissues caused by the production, by the immune system, of the body's own antibodies, or for suppressing rejection during, e.g., tissue or organ transplantation. Compounds which inhibit cell proliferation are useful for treating conditions characterized by cell hyperproliferation, e.g., as antineoplastic agents. Compounds of the invention are also useful as research tools, e.g., to investigate cell signaling.

In accordance with a preferred embodiment, the present invention includes methods of treating patients suffering from depressed immune systems resulting from, e.g., chemotherapy treatment, radiation treatment, radiation sickness, or HIV/AIDs; conditions associated with primary B-cell deficiency (such as, e.g., Bruton's congenital a-(-globulinemia or common variable immunodeficiency) or primary T-cell deficiency (such as, e.g., the DiGeorge and Nezelof syndromes, ataxia telangiectasia or Wiskott-Aldrich syndrome); severe combined immunodeficiency (SCID), etc.; with an immunostimulant of the invention. The immunostimulants can also be used for vaccines (e.g., anti-bacterial, anti-fungal, anti-viral or anti-protozoiasis), particularly for patients having immunocompromised states; or for anti-neoplastic vaccines.

In another preferred embodiment, the invention includes methods of treating patients suffering from autoimmune disorders, such as, e.g., rheumatoid arthritis, glomerulonephritis, Hashimoto's thyroiditis, multiple sclerosis, T cell leukemia, systemic lupus erythematosus, myasthenia gravis, autoimmune hemolytic anemia, autoimmune thrombocytopenic purpura, type 1 diabetes, Chrohn's disease, Grave's disease, celiac disease, or the like, with an immunosuppressant of the invention. Immunosuppressants of the invention are also useful for treating tissue or organ transplant rejection, e.g., hyper-acute or chronic graft-vs-host disease, allograft or xenograft rejection, etc.

As mentioned, the compounds of the invention also inhibit hyperproliferation of cells, e.g., they can exhibit anti-neoplastic activity. As a result, the inventive compounds are useful in the treatment of a variety of conditions, e.g. cancers involving T cells and B cells. Among the types of cancer which can be treated with compounds of the invention are e.g., leukemias, lymphomas, ovarian cancer and breast cancer.

Compounds of the invention can be attached to an agent that, e.g., targets certain tumors, such as an antibody which is specific for a tumor-specific antigen. In this manner, compounds of the invention can be transported to a target cell in which they then can act. The compounds can be further attached to a conventional cytotoxic agent (such as a toxin or radioactivity). When the inventive molecule binds to its target, e.g., p56^{lck}, it not only will inhibit the enzymatic activity, but will also destroy the target, and/or the cell in which the target resides, by means of the toxin.

The preferred aspects include pharmaceutical compositions comprising a compound of this invention and a pharmaceutically acceptable carrier and, optionally, another active agent as discussed below; a method of inhibiting or stimulating a p56^{lck} kinase, e.g., as determined by a conventional assay or one described herein, either in vitro or in vivo (in an animal, e.g., in an animal model, or in a mammal or in a human); a method of modulating an immune response, e.g., enhancing or inhibiting an immune reaction; a method of treating a disease state, e.g., an autoimmune disease, a neoplasm, etc.; a method of treating a disease state modulated by p56^{lck} kinase activity, in a mammal, e.g., a human, including those disease conditions mentioned herein.

The present invention also relates to useful forms of the compounds as disclosed herein, such as pharmaceutically acceptable salts and prodrugs of all the compounds of the present invention. Pharmaceutically acceptable salts include those obtained by reacting the main compound, functioning as a base, with an inorganic or organic acid to form a salt, for example, salts of hydrochloric acid, sulfuric acid, phosphoric acid, methane sulfuric acid, camphor sulfonic acid, oxalic acid, maleic acid, succinic acid and citric acid.

Pharmaceutically acceptable salts also include those in which the main compound functions as an acid and is reacted with an appropriate base to form, e.g., sodium, potassium, calcium, magnesium, ammonium, and chlorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropiionates, picrates, pivalates, propionates, succinates, tartrates, thiocyannates, tosylates, mesylates and undecanoates.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

The compounds of the invention can be administered alone or as an active ingredient of a formulation. Thus, the present invention also includes pharmaceutical compositions of a compound of the invention or a salt thereof, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Isol, editor), 1553-1593 (current edition).

In view of their high degree of selective p56^{lck} kinase inhibition or stimulation, the compounds of the present invention can be administered to anyone requiring p56^{lck} kinase inhibition or stimulation. Administration may be accomplished according to patient needs, for example, orally, nasally, parenterally (subcutaneously, intravenously, intramuscularly, intrasternally, and by infusion) by inhalation, rectally, vaginally, topically and by ocular administration. Injection can be, e.g., intramuscular, intraperitoneal, intravenous, etc.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time-release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The compounds can be administered as the sole active agent or in combination with other pharmaceutical agents, such as other agents which inhibit or stimulate tyrosine kinases, signal transduction processes, cell proliferation and/or immune responses. Inhibitory agents include, e.g., cyclosporine, FK506, rapamycin, leflunomide, butenamindes, corticosteroids, atomeric acid, dipeptide derivative, tyrphostin, Doxorubicin or the like. In such combinations, each active ingredient can be administered either in accordance with its usual dosage range or a dose below its usual dosage range.

The dosages of the compounds of the present invention depend upon a variety of factors including the particular syndrome to be treated, the severity of the symptoms, the age, sex and physical condition of the patient, the route of administration, the frequency of the dosage interval, the particular compound utilized, the efficacy, toxicology profile, pharmacokinetic profile of the compound, and the presence of any deleterious side-effects, among other considerations.

By “effective dose” or “therapeutically effective dose” is meant herein, in reference to the treatment of a cancer, an amount sufficient to bring about one or more of the following results: reduce the size of the cancer; inhibit the metastasis of the cancer; inhibit the growth of the cancer, preferably stop cancer growth; relieve discomfort due to the cancer; and prolong the life of a patient inflicted with the cancer.

A “therapeutically effective amount,” in reference to the treatment of a hyper-proliferative cell disorder other than a cancer refers to an amount sufficient to bring about one or more of the following results: inhibit the growth of cells causing the disorder, preferably stopping the cell growth; relieve discomfort due to the disorder; and prolong the life of a patient suffering from the disorder.

A “therapeutically effective amount”, in reference to treatment of an autoimmune disorder refers to an amount sufficient to bring about one or more of the following results: inhibit or ameliorate the symptoms of the disease; inhibit progressive degeneration of cells involved in the disorder; relieve discomfort due to the disorder; and prolong the life of a patient suffering from the disorder.

A “therapeutically effective amount”, in reference to treatment of a patient undergoing tissue or organ transplantation refers to an amount sufficient to bring about one or more of the following results: inhibit or prevent rejection of the transplanted material; relieve discomfort resulting from rejection of the transplant; and prolong the life of a patient receiving a transplant.

A “therapeutically effective amount,” in reference to treatment of an immunosuppressive patient refers to an amount sufficient to bring about one or more of the following results: increase the number of T cells or number of activated T cells; reduce the immuosuppressed state of the patient; relieve discomfort due to the disorder; and prolong the life of a patient suffering from the disorder.

The compounds of the invention are administered at dosage levels and in a manner customary for p56^{lck} kinase inhibitors or stimulators, or other analogous drugs, such as those mentioned above. For example, cyclosporine is administered (for transplants) at about 7.95±2.81 mg/kg/day (see PDR(Physician's Desk Reference)); FK506 is administered (for transplants) at about 0.15-0.30 mg/kg/day (see PDR); and rapamycin is administered (for transplants) at about 2-6 mg/day, e.g., about 0.024 mg/kg/day for an 81 kg adult (see Thomas A. Stargy Transplantation Institute web site). See also, e.g., disclosures in U.S. Pat. Nos. 5,688,824, 5,914,343, 5,217,999, 6,133,301 and publications cited therein.

For example, compounds of the invention or a salt thereof, can be administered, in single or multiple doses, at a dosage level of, for example, 1 μg/kg to 500 mg/kg of body weight of patient/day, preferably between about 100 μg/kg/day and 25 mg/kg/day. Dosages can be adjusted so as to generate an immunostimulatory or immunosuppressive effect, as desired. A lower dosage (immunostimulatory) can be between about 1 μg/kg/day and 750 μg/kg/day, preferably between about 10 μg/kg/day and 500 mg/kg/day. A higher dosage (immunosuppressive) can be between about 1 mg/kg/day and 750 mg/kg/day, preferably between about 10 mg/kg/day and 450 mg/kg/day.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

In the foregoing and in the following examples, all temperatures are set forth in degrees Celsius; and, unless otherwise indicated, all parts and percentages are by weight.

### EXAMPLES

### Assay Example 1

Compounds of the invention were obtained from commercial sources and were tested using a high-throughput Enzyme Immunoassay (EIA) developed to rapidly quantify inhibition.

Biological activities were measured using said EIA assay using 96 well medium binding EIA plates (Costar). Wells were coated with 100 μl of human CD3 ζ chain ITAM 2 phosphopeptide conjugated to BSA (˜10 pmole peptide equivalent) in PBS overnight at 4° C. and blocked with 300 μl of PBS containing 5% (wt/vol) powdered skim milk for 1 h at 37° C. After washing with PBS containing 1% Tween 20 (PBST), 3 times, 100 μl of precalibrated bacterial lysate containing recombinant GST Lck SH2 domain fusion protein was added in the presence or absence of test compounds and incubated for 1 h at room temperature. After 3 extensive washings with PBST, 100 μl of PBS containing HRP-conjugated rabbit anti-GST antibody was added and incubated for 1 h. After extensive washing with PBST, 100 μl of TMB substrate was added and absorbance at 620 nm was measured using a multiwell EIA plate reader (Anthos HTIII). The percent inhibition (% inhibition) was calculated based on the optical density (OD) using the formula: % inhibition=100−(ΔOD of test well/ΔOD of positive wells)×100, where ΔOD was calculated by subtracting background OD (average OD of negative wells) from the test as well as positive control wells. Error analysis was performed on two or more measurements.

The results from this solid phase EIA inhibition assays for compounds according to the invention are presented in FIGS. 1-4.

### Structure-Activity Relationships Example 1

Not wishing to be bound by theory at all, development of structure-activity relationships (SAR)/pharmacophores for compounds 276-0 to 276-20 are useful in facilitating a lead optimization process (compound 276-0 corresponds to compound 276 from U.S. application Ser. No. 10/582,640). Such a SAR may be ligand based where only the structures of the ligands themselves are considered. Alternatively, a target-based pharmacophore can be developed via, for example, docking studies of all the similar compounds from which functional groups of importance on both the compounds and the target molecule can be identified.

Compounds 276-0 to 276-20 were placed into two groups, i.e., groups A and B. The compounds in group A are those with >60% inhibitory activity and in group B are those with less that 60% inhibitory activity. (These values were obtained in assay example 1 as discussed herein.)

Common to all the compounds is an amide linkage attached to a central five-member heterocycle and to an aromatic ring. The amide's carbonyl group is attached to the 5-membered ring's nitrogen and the amide's nitrogen is attached to the aromatic ring. The highly active compounds all contain a furan ring linked via a double bond to the heterocycle, which is then linked to an aromatic ring that contains an acid group in the meta or para position. The only exception is 276-11, which has a phenol moiety with the hydroxyl in the ortho position rather than the furan ring; however, this compound has the lowest activity among the highly active compounds. In the lower activity compounds, which are in group B, the furan ring is omitted, with the exception of 276-5 and 276-8. Many of the low activity compounds contain benzoic acid moieties, though in most cases the furan is omitted or exchanged with pyrrole ring, which lacks the hydrogen bond acceptor of the furan. In addition, 276-5 and 276-8 contain ester moieties versus the acid on the terminal phenyl ring, which may also contribute to the decreased activity. The methyl group could be causing steric hindrance or the lack of the negative charge could be affecting the binding. Interesting are 276-(6, 14, and 15), which all contain a phenol group and lack the furan moiety, as in 276-11. In these lower activity compounds the hydroxyl is meta or para versus ortho in the more active compound. This motif suggests that the hydroxyl in 276-11 may act as an acceptor, replacing that in the furan ring in the other more active compounds. Overall, these results indicate that beyond the heterocycle-amide-phenyl ring central core of the compounds 276-0 to 276-20 the presence of a furan ring 1,3 linked to benzoic acid moiety facilitates activity, though alternate functional groups with acceptor moieties may be considered to enhance the inhibitory activity.

Alternatively, the availability of the similar compounds can be used to identify interesting interactions between the inhibitors and the target protein. To identify relevant drug-protein interactions all the active compounds can be docked into the putative bonding site of the protein with the resulting structures examined collectively to identify consensus interactions. Such consensus interactions may be assumed to be more representative of the experimental regimen versus the interactions observed for a single docked molecule. Compounds 276-0 to 276-20 were examined comparing the interactions between the set of stronger inhibitors (>60% inhibition) and the set of weaker inhibitors (<60% inhibition) to provide insight into the development of a target-based pharmacophore. Pair-wise interactions of 3.0 Å or less between the protein and all ligand atoms were considered in the determination of relevant protein residues. Residues which had at least five of these close interactions with ligand atoms were: Arg134, Lys179, His180, Tyr181, Lys182, Arg184, Ile193, Ser194, Gly215, Leu216, and Cys217 as shown in FIG. 5. These residues are in the BG and EF loops and βD strand of the Lck SH2 domain.

FIG. 6, summarizes the hydrogen bonds formed between the ligands associated with 276 and the protein in the docked conformations. One obvious difference between the strong and weak inhibitors is that strong inhibitors make more hydrogen bonds with the protein, usually through the carboxylic acid. Another difference between the two sets of compounds is that they interact with different protein residues. Residues Lys179, Lys182, and Arg184 make more hydrogen bonds with the strong inhibitors while residues Arg134 and Arg184 hydrogen bond to the weak inhibitors, revealing different binding modes. The acid can interact with the same residue, Arg134 for some weak inhibitors, or with two different residues, Arg184 and Lys182 for some strong inhibitors.

FIG. 7 in parts A and B show example binding modes of a strong and weak inhibitor, respectively. The predicted binding conformation of 276-13 illustrates an orientation common among several of the stronger inhibitors in which the compounds interact closely with Arg184 and Lys182. Alternatively, compound 276-8 illustrates the binding orientation common among several of the weak inhibitors that allows close interaction with Arg134.

The fact that docked compounds do not all have perfectly superimposed orientations may be due to a variety of reasons, e.g., their chemical structure and size, the lack of a very well defined cavity or groove adjacent to the pY+3 hydrophobic cavity, and the inherent limitations of the docking method. In spite of this, some predictions can be made based on frequent occurrences of common binding motifs. The ability of a compound to adopt a favorable binding conformation in which it can hydrogen bond to Lys179, Lys182, and Arg184 seems strongly related to its activity.

Applying this approach with the compounds 276-0 to 276-20 lead to the identification of residues Lys179, Lys182, and Arg184 of the Lck SH2 domain as being important for inhibitor-receptor interaction, which interaction is not limited to the use of compounds 276-0 to 276-20.

Additionally, compounds of the invention can be subjected to various other tests, e.g., ones described or cited herein, and also to tests described in more detail in the assay examples of U.S. application Ser. No. 10/582,640, which is incorporated herein by reference.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited herein are hereby incorporated in their entirety by reference.

### BRIEF DESCRIPTION OF DRAWINGS

FIG. 1-4. Illustrates experimental inhibition values for compounds of the invention. The results are expressed as mean±standard deviation inhibition of at least two experiments.

FIG. 5. Illustrates a detailed view of Lck residues which have frequent close contacts (<3 Å) with the predicted docked conformations of compounds 276-0 to 276-20.

FIG. 6. Illustrates hydrogen bonds between docked compounds and protein residues for compounds 276-0 to 276-20.

FIG. 7. Illustrates docked conformations of a strong and a weak inhibitor from the compounds 276-0 to 276-20. Compounds are shown in colored ball and stick representation. Lck protein is shown as a cartoon except those residues that form hydrogen bonds to the compounds which are shown in grey ball and stick representation.

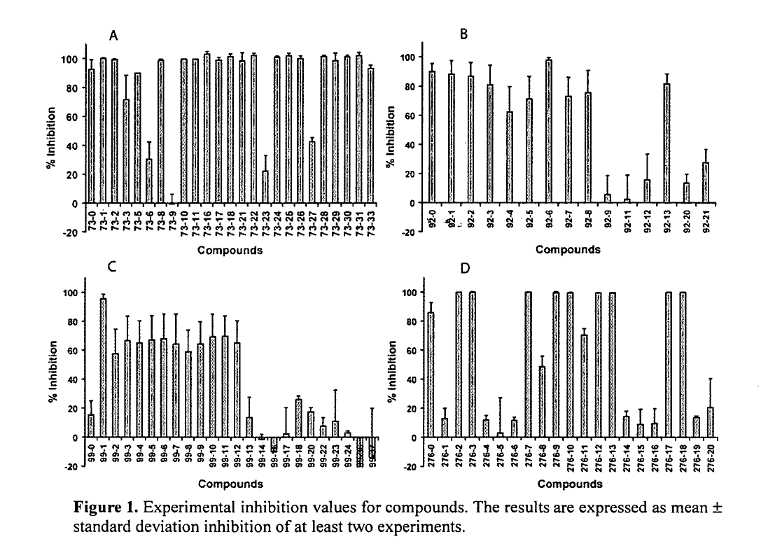

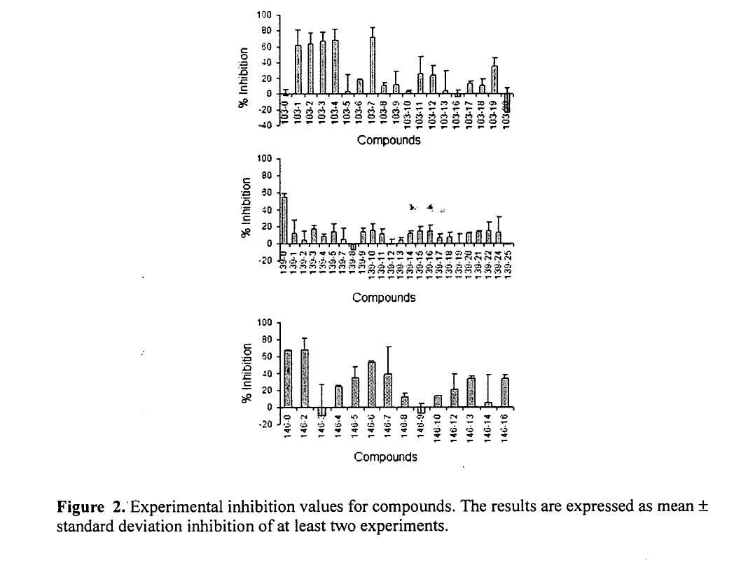

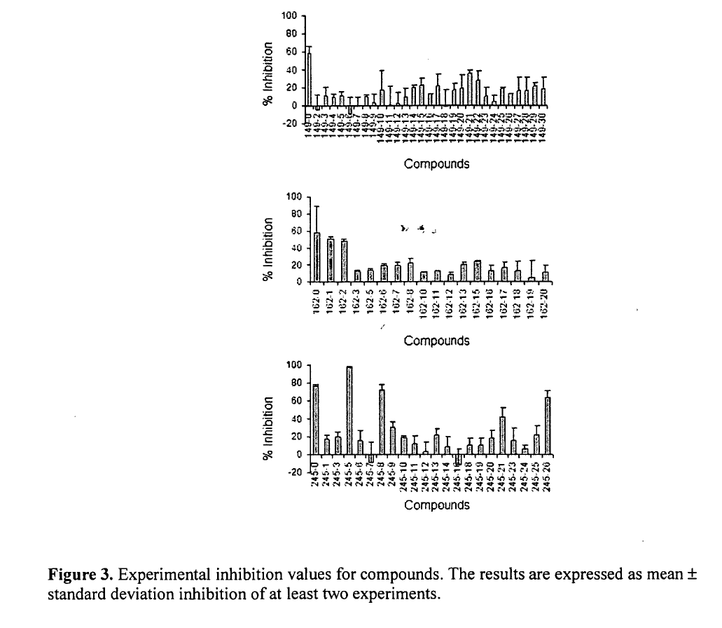

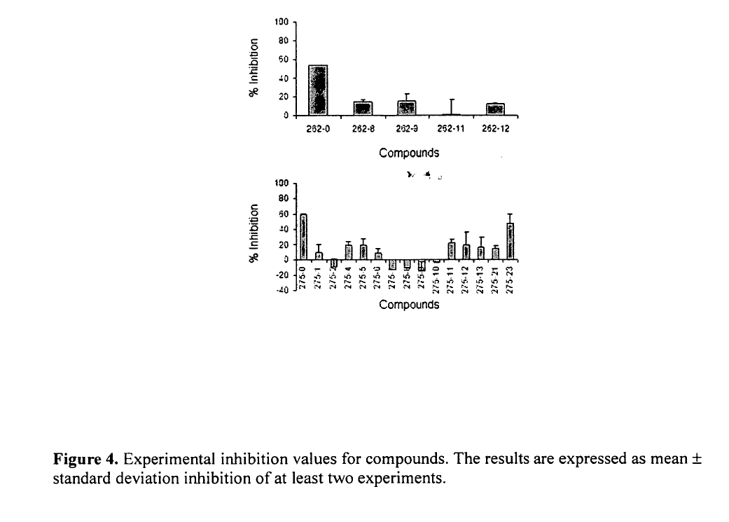

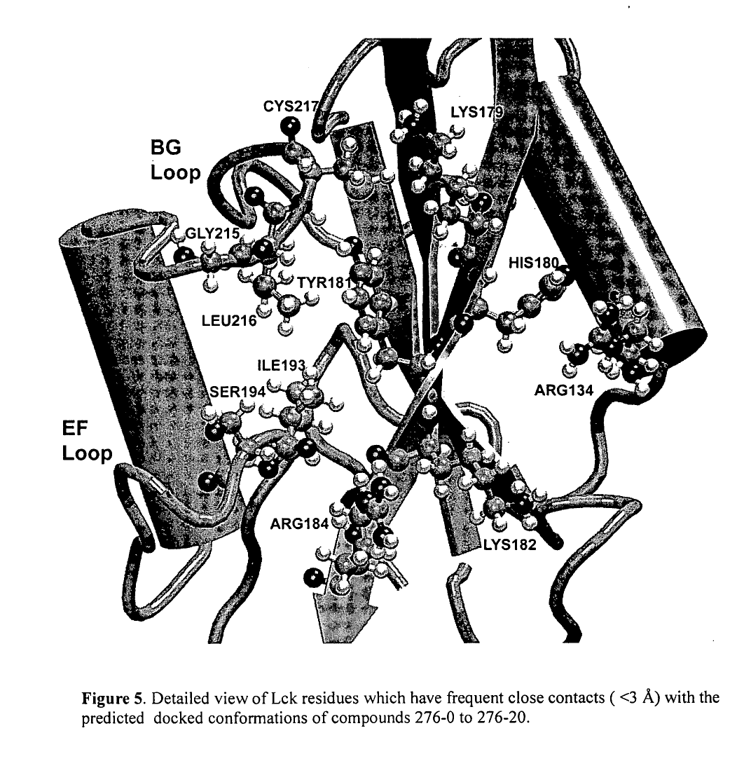

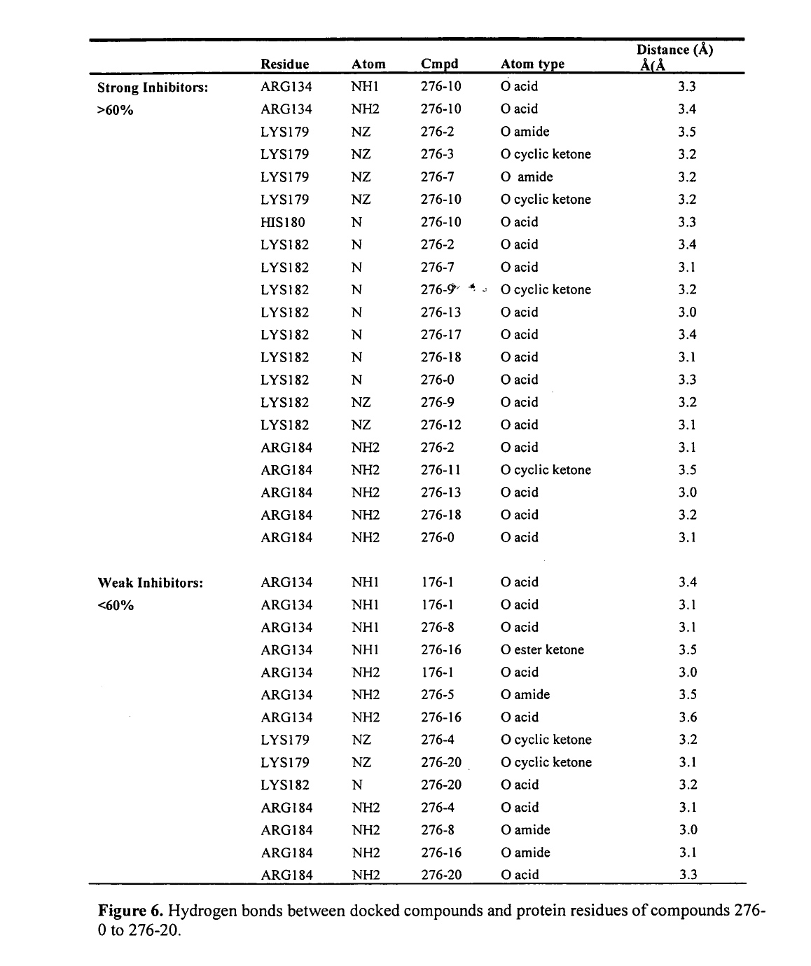

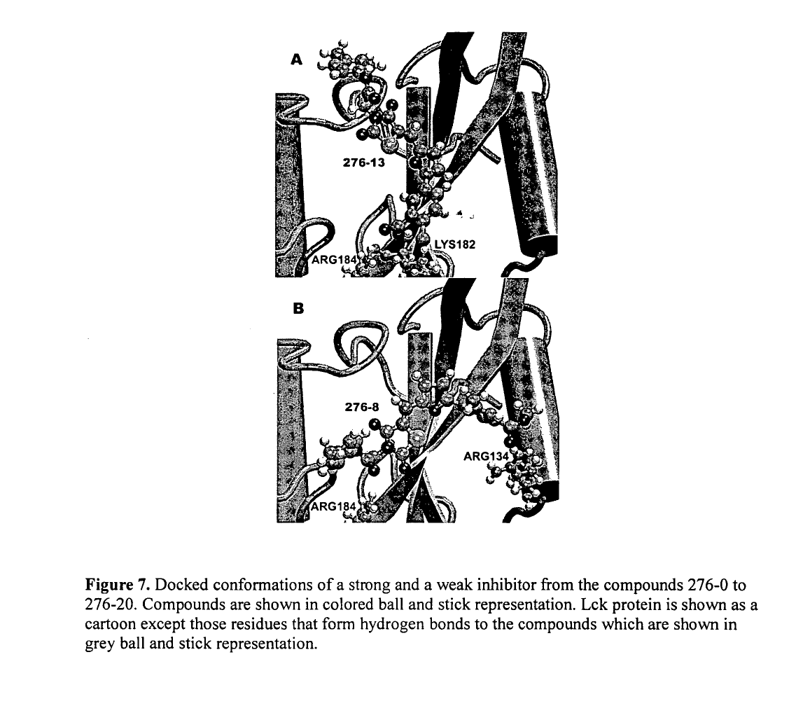

### REFERENCES

(1) Tong, L.; Warren, T. C.; King, J.; Betageri, R.; Rose, J.; Jakes, S. Crystal structures of the human p56lck SH2 domain in complex with two short phosphotyrosyl peptides at 1.0 A and 1.8 A resolution. J. Mol. Biol. 1996, 256, 601-610.
(2) Broadbridge, R. J.; Sharma, R. P. The Src Homology-2 Domains (SH2 domains) of the protein tyrosine kinase p56lck: structure, mechanism and drug design. Current Drug Targets 2000, 1, 365-386.
(3) Straus, D. B.; Weiss, A. Genetic evidence for the involvement of the lck tyrosine kinase in signal transduction through the T cell antigen receptor. Cell 1992, 70, 585-593.
(4) Weiss, A.; Littman, D. R. Signal transduction by lymphocyte antigen receptors. Cell. 1994, 76, 263-274.
(5) Straus, D. B.; Chan, A. C.; Patai, B.; Weiss, A. SH2 domain function is essential for the role of the Lck tyrosine kinase in T cell receptor signal transduction. J. Biol. Chem. 1996, 271, 9976-9981.
(6) Pawson, T.; Gish, G. D. SH2 and SH3 domains: from structure to function. Cell. 1992, 71, 359-362.
(7) Cody, W. L.; Lin, Z.-W.; Panek, R. L.; Rose, D. W.; Rubin, J. R. Progress in the development of inhibitors of SH2 domains. Current Pharmaceutical Deesign 2000, 6, 59-98.
(8) Cousins-Wasti, R.; Ingraham, R. H.; Morelock, M. M.; Grygon, C. A. Determination of affinities for lck SH2 binding peptides using a sensitive fluorescence assay: comparison between the pYEEIP and pYQPQP consensus sequences reveals context-dependent binding specificity. Biochemistry. 1996, 35, 16746-16752.
(9) Sawyer, T. K. Src homology-2 domains: structure, mechanisms, and drug discovery. Biopolymers. 1998, 47, 243-261.
(10) Beaulieu, P. I.; Cameron, D. R.; Ferland, J. M.; Gauthier, J.; Ghiro, E.; Gillard, J.; Gorys, V.; Poirier, M.; Rancourt, J.; Wernic, D.; Llinas-Brunet, M.; Betageri, R.; Cardozo, M.; Hickey, E. R.; Ingraham, R.; Jakes, S.; Kabcenell, A.; Kirrane, T.; Lukas, S.; Patel, U.; Proudfoot, J.; Sharma, R.; Tong, L.; Moss, N. Ligands for the tyrosine kinase p56lck SH2 domain: discovery of potent dipeptide derivatives with monocharged, nonhydrolyzable phosphate replacements. J. Med. Chem. 1999, 42, 1757-1766.
(11) Lee, T., R.; Lawrence, D. S. SH2-directed ligands of the Lck tyrosine kinase. J. Med. Chem. 2000, 43, 1173-1179.
(12) Kuriyan, J.; Cowburn, D. Modular peptide recognition domains in eukaryotic signaling. Annu. Rev. Biophys. Biomol. Struct. 1997, 26, 259-288.
(13) Songyang, Z.; Cantley, L. C. Recognition and specificity in protein tyrosine kinase-mediated signalling. Trends Biochem. Sci. 1995, 20, 470-475.
(14) Marengere, L.; Songyang, Z.; Gish, G. D.; Schaller, M. D.; Parsons, J. T.; Stem, M. J.; Cantley, L. C.; Pawson, T. SH2 domain specificity and activity modified by a single residue. Nature. 1994, 369, 502-505.
(15) Songyang, Z.; Gish, G.; Mbamalu, G.; Pawson, T.; Cantley, L. C. A single point mutation switches the specificity of group III Src homology (SH2) domains to that of group I SH2 domains. J. Biol. Chem. 1995, 270, 26029-26032.
(16) Brennan, M. B. Drug Discovery Filtering Out Failures Early in the Game. Chemical & Engineering News 2000, 78, 63-73.
(17) Estrada, E.; Uriarte, E.; Montero, A.; Teijeira, M.; Santana, L. D.; Clercq, E. A novel approach for the virtual screening and rational design of anticancer compounds. J. Med. Chem. 2000, 43, 1975-1985.
(18) Baxter, C. A.; Murray, C. W.; Waszkowycz, B.; Li, J.; Sykes, R. A.; Bone, R. G.; Perkins, T. D.; Wylie, W. New approach to molecular docking and its application to virtual screening of chemical databases. J. Chem. Inf. Comput. Sci. 2000, 40, 254-262.
(19) Walters, W. P.; Stahl, M. T.; Murcko, M. A. Virtual screening—an overview. Drug Discov. Today 1998, 3, 160-178.
(20) Massova, I.; Martin, P.; Bulychev, A.; Kocz, R.; Doyle, M.; Edwards, B. F.; Mobashery, S. Templates for design of inhibitors for serine proteases: application of the program DOCK to the discovery of novel inhibitors for thrombin. Bioorg. Med. Chem. Lett. 1998, 8, 2463-2466.
(21) Li, S.; Gao, J.; Satoh, T.; Friedman, T. M.; Edling, A. E.; Koch, U.; Choksi, S.; Han, X.; Korngold, R.; Huang, Z. A computer screening approach to immunoglobulin superfamily structures and interactions: discovery of small non-peptidic CD4 inhibitors as novel immunotherapeutics. Proc. Natl. Acad. Sci. USA. 1997, 94, 73-78.
(22) Chen, I.-J.; Neamati, N.; Nicklaus, M. C.; Orr, A.; Anderson, L.; Barchi, J. J. J.; Kelley, J. A.; Pommier, Y.; MacKerell, A. D. J. Identification of HIV-1 integrase inhibitors via three-dimensional database searching using ASV and HIV-1 integrases as targets. Bioorg. Med. Chem. 2000, 8, 2385-2398.
(23) Deborah, A. L.; Willian, V. M.; Linda, K. J. Application of Virtual screening tools to a protein-protein interaction: database mining studies on the growth hormone. Med. Chem. Res. 1999, 9, 579-591.
(24) Pan, Y.; Huang, N.; Cho, S.; MacKerell, A. D., Jr. Consideration of Molecular Weight during Compound Selection in Virtual Target-Based Database Screening. J. Chem. Inf. Comput. Sci. 2003, 43, 267-272.
(25) SYBYL; 6.7 ed.; Tripos Associates: St. Louis. Mo.
(26) Gasteiger, J.; Rudolph, C.; Sadowski, J. Automatic generation of 3D-atomic coordinates for organic molecules. Tetrahedron Comput. Methodol. 1990, 3, 537.
(27) Berman, H. M.; Westbrook, J.; Feng, Z.; Gilliland, G.; Bhat, T. N.; Weissig, H.; Shindyalov, I. N.; Bourne, P. E. The Protein Data Bank. Nucleic Acids Research 2000, 28, 235-242.
(28) Meng, E. C.; Shoichet, B. K.; Kuntz, I. D. Automated docking with grid-based energy evaluation. J. Comput. Chem. 1992, 13, 505-524.
(29) Leach, A. R.; Kuntz, I. D. Conformational analysis of flexible ligands in macromolecular receptor sites. J. Comput. Chem. 1992, 13, 730-748.
(30) Connolly, M. L. Solvent-accessible surfaces of proteins and nucleic acids. Science 1983, 221, 709-713.
(31) Ferrin, T. E.; Huang, C. C.; Jarvis, L. E.; Langridge, R. The MIDAS display system. J. Mol. Graphics 1988, 6,13-27.
(32) Goodford, P. J. A computational procedure for determining energetically favorable binding sites on biologically important macromolecules. J. Med. Chem. 1984, 28, 849-857.
(33) Ewing, T. J. A.; Kuntz, I. D. Critical evaluation of search algorithms used in automated molecular docking. J. Comput. Chem. 1997, 18, 1175-1189.
(34) Oprea, T. I. Property distribution of drug-related chemical databases. J. Comput. Aided Mol. Des. 2000, 14, 251-264.
(35) Oprea, T. I.; Davis, A. M.; Teague, S. J.; Leeson, P. D. Is There a Difference between Leads and Drugs? A Historical Perspective. J. Chem. Inf. Comput. Sci. 2001, 41, 1308-1315.
(36) Teague, S. J.; Davis, A. M.; Leeson, P. D.; Oprea, T. I. The design of leadlike combinatorial libraries. Angew. Chem., Int. Ed. 1999, 38, 3743-3748.
(37) Godden, J. W.; Xue, L.; Bajorath, J. Combinatorial preferences affect molecular similarity/diversity calculations using binary fingerprints and Tanimoto coefficients. J. Chem. Inf. Comput. Sci. 2000, 40, 163-166.
(38) MOE; 2002 ed.; Chemical Computing Group Inc.: Montreal, Quebec, Canada.
(39) Lipinski, C. A.; Lombardo, F.; Dominy, B. W.; Feeney, P. J. Experimental and computational approaches to estimate solubility and permeability in drug discovery development settings. Adv. Drug Delivery Research 1997, 23.
(40) Su, A-L.; Lorber, D. M.; Weston, G. S.; Baase, W. A.; Matthew, B. W.; Schoichet, B. K. Docking molecules by families to increase the diversity of hits in database screening: computational strategy and experimental evaluation. Proteins 2001, 42, 279-293.
(41) Kubinyi, H. Structure-based design of enzyme inhibitors and receptor ligands. Current Opinion in Drug Discovery and Development 1998, 1, 4-15.
(42) Shoichet, B. K.; Leach, A. R.; Kuntz, I. D. Ligand salvation in molecular docking. PROTEINS: Structure, Function and Genetics. 1999, 34, 4-16.
(43) Payne, G.; Stolz, L. A.; Pei, D.; Band, H.; Shoelson, S. E.; Walsh, C. T. The phosphopeptide-binding specificity of Src family SH2 domains. Chemistry and Biology 1994, 1, 99-105.
(44) Songyang, Z.; Shoelson, S. E.; Chaudhuri, M.; Gish, G.; Pawson, T.; Haser, W. G.; King, F.; Roberts, T.; Ratnofsky, S.; Lechleider, R. J.; Neel, B. G.; Birge, R B.; Fajardo, J. E. F.; Chou, M. M.; Harafusa, H.; Schaffhausen, B.; Cantley, L. C. SH2 domains recognize specific phosphopeptide sequences. Cell 1993, 72, 767-778.
(45) Liu, X.; Brodeur, S. R.; Gish, G.; Songyang, Z.; Cantley, L. C.; Laudano, A. P.; Pawson, T. Regulation of c-Src tyrosine kinase activity by the Src SH2 domain. Oncogene 1993, 8, 1119-1126.
(46) Humphrey, W.; Dalke, A.; Schulten, K. VMD—visual molecular dynamics. J. Mol. Graphics 1996, 14, 33-38.
   And the following additional references are also of interest:

Johnson, M. A.; Maggiora, G. M., Concepts and Applications of Molecular Similarity. Wiley: N.Y., 1990.

Willett, P., Similarity and Clustering Techniques in Chemical Information Systems. Research Studies Press: Letchworth, 1987.

Dean, P. M., Molecular Similarity in Drug Design. Chapman & Hall: Glasgow, 1994.

Van Drie, J. H.; Lajiness, M. S., Approaches to virtual library design. Drug Discovery Today 1998, 3, (6), 274-283.

Martin, Y. C.; Kofron, J. L.; Traphagen, L. M., Do structurally similar molecules have similar biological activity? J. Med. Chem. 2002, 45, (19), 4350-4358.

Shanmugasundaram, V.; Maggiora, G. M.; Lajiness, M. S., Hit-directed nearest-neighbor searching. J. Med. Chem. 2005, 48, (1), 240-248.

Matter, H., Selecting optimally diverse compounds from structure databases: A validation study of two-dimensional and three-dimensional molecular descriptors. J. Med. Chem. 1997, 40, (8), 1219-1229.

Willett, P.; Winterman, V.; Bawden, C., Cluster Analysis Methods in Chemical Information Systems: Selection of Compounds for Biological Testing and Clustering of Substructure Search Output. J. Chem. Inf. Comput. Sci. 1986, 26, 109-118.

Huang, N.; Nagarsekar, A.; Xia, G. J.; Hayashi, J.; MacKerell, A. D., Identification of non-phosphate-containing small molecular weight inhibitors of the tyrosine kinase p56 Lck SH2 domain via in silico screening against the pY+3 binding site. J. Med. Chem. 2004, 47, (14), 3502-3511.

Todeschini, R.; Consonni, V., Handbook of Molecular Descriptors, In Methods and Principles in Medicinal Chemistry. Wiley-VCH: New York, 2000; Vol. 11, p 667.

Bajorath, J., Selected concepts and investigations in compound classification, molecular descriptor analysis, and virtual screening. J. Chem. Inf. Comput. Sci. 2001, 41, (2), 233-245.

Bender, A.; Glen, R. C., Molecular similarity: a key technique in molecular informatics. Organic & Biomolecular Chemistry 2004, 2, (22), 3204-3218.

Brown, R. D.; Martin, Y. C., The information content of 2D and 3D structural descriptors relevant to ligand-receptor binding. J. Chem. Inf. Comput. Sci. 1997, 37, (1), 1-9.

Martin, Y. C.; Willett, P.; Lajiness, M.; Johnson, M.; Maggiora, G.; Martin, E.; Bures, M. G.; Gasteiger, J.; Cramer, R. D.; Pearlman, R. S.; Mason, J. S., Diverse viewpoints on computational aspects of molecular diversity. J. Combinatorial Chem. 2001, 3, (3), 231-250.

Horvath, D.; Jeandenans, C., Neighborhood behavior of in silico structural spaces with respect to in vitro activity spaces—A novel understanding of the molecular similarity principle in the context of multiple receptor binding profiles. J. Chem. Inf. Comput. Sci. 2003, 43, (2), 680-690.

Willett, P.; Barnard, J. M.; Downs, G. M., Chemical similarity searching. J. Chem. Inf. Comput. Sci. 1998, 38, (6), 983-996.

Unity Unity Chemical Information Software, Tripos Associates: St. Louis, Mo., 1996.

Brown, R. D.; Martin, Y. C., An evaluation of structural descriptors and clustering methods for use in diversity selection. Sar And Qsar In Environmental Research 1998, 8, (1-2), 23-39.

Kuntz, I. D.; Blaney, J. M.; Oatley, S. J.; R., L.; Ferrin, T. E., A geometric approach in macromolecule-ligand interactions. J. Mol. Biol. 1982, 161, 269-288.

Ewing, T. J. A.; Kuntz, I. D., Critical evaluation of search algorithms for automated molecular docking and database screening. J. Comput. Chem. 1997, 18, (9), 1175-1189.

Pan, Y. P.; Huang, N.; Cho, S.; MacKerell, A. D., Consideration of molecular weight during compound selection in virtual target-based database screening. J. Chem. Inf. Comput. Sci. 2003, 43, (1),267-272.

Chen, I. J.; Neamati, N.; Nicklaus, M. C.; Orr, A.; Anderson, L.; Barchi, J. J.; Kelley, J. A.; Pommier, Y.; MacKerell, A. D., Identification of HIV-1 integrase inhibitors via three-dimensional database searching using ASV and HIV-1 integrases as targets. Bioorgan Med Chem 2000, 8, (10), 2385-2398.

Lipinski, C. A.; Lombardo, F.; Dominy, B. W.; Feeney, P. J., Experimental and computational approaches to estimate solubility and permeability in drug discovery and development settings. Advanced Drug Delivery Reviews 1997, 23, (1-3), 3-25.

MOE Molecular Operating Environment, Montreal, Canada, 2002.

Brooks, B. R.; Bruccoleri, R. E.; Olafson, B. D.; States, D. J.; Swaminathan, S.; Karplus, M., CHARMM: A Program for Macromolecular Energy, Minimization, and Dynamics Calculations. J. Comput. Chem. 1983, 4, 187-217.

Pettersen, E. F.; Goddard, T. D.; Huang, C. C.; Couch, G. S.; Greenblatt, D. M.; Meng, E. C.; Ferrin, T. E., UCSF chimera—A visualization system for exploratory research and analysis. J. Comput. Chem. 2004, 25, (13), 1605-1612.

Mills, J. E. J.; Dean, P. M., Three-dimensional hydrogen-bond geometry and probability information from a crystal survey. Journal of Computer-Aided Molecular Design 1996, 10, (6), 607-622.

Klein, C.; Kaiser, D.; Kopp, S.; Chiba, P.; Ecker, G. F., Similarity based SAR (SIBAR) as tool for early ADME profiling. J. Computer-Aided Molecular Design 2002, 16, (11), 785-793.

Kibbey, C.; Calvet, A., Molecular property eXplorer: A novel approach to visualizing SAR using tree-maps and heatmaps. J. Chem. Inf. Mod. 2005, 45, (2), 523-532.

Bohm, M.; Sturzebecher, J.; Klebe, G., Three-dimensional quantitative structure-activity relationship analyses using comparative molecular field analysis and comparative molecular similarity indices analysis to elucidate selectivity differences of inhibitors binding to trypsin, thrombin, and factor Xa. J. Med. Chem. 1999, 42, (3), 458-477.

Sheridan, R. P.; Feuston, B. P.; Maiorov, V. N.; Kearsley, S. K., Similarity to molecules in the training set is a good discriminator for prediction accuracy in QSAR. J. Chem. Inf. Comput. Sci. 2004, 44, (6), 1912-1928.

TABLE 1

| | | | |
|---|---|---|---|
| 1 | 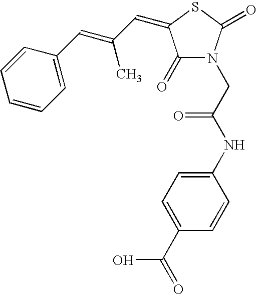 | C22H18N2O5S | 276-1 |
| 2 | 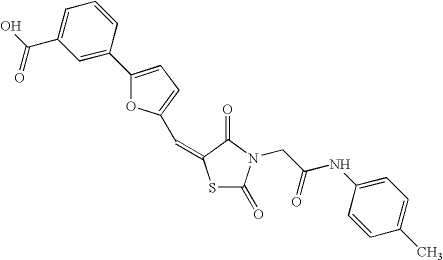 | C24H18N2O6S | 276-2 |
| 3 | 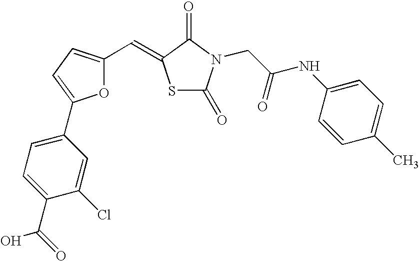 | C24H17ClN2O6S | 276-3 |
| 4 | 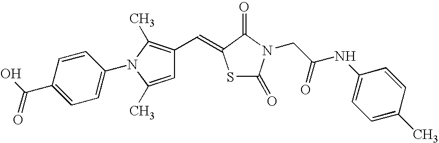 | C26H23N3O5S | 276-4 |
| 5 | 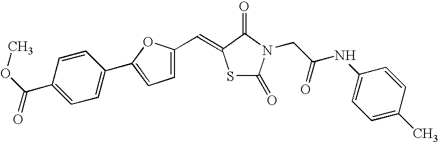 | C25H20N2O6S | 276-5 |
| 6 | 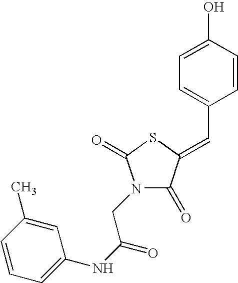 | C19H16N2O4S | 276-6 |
| 7 | 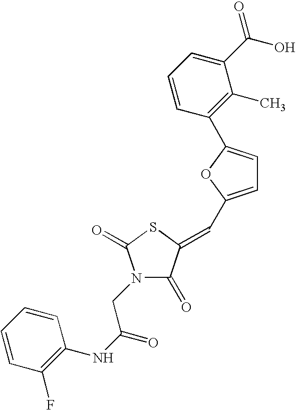 | C24H17FN2O6S | 276-7 |
| 8 | 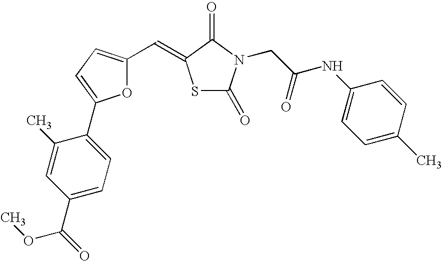 | C26H22N2O6S | 276-8 |
| 9 | 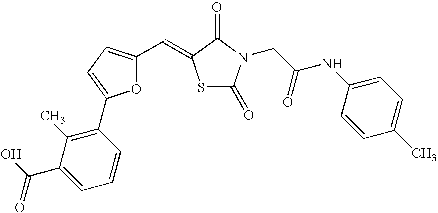 | C25H20N2O6S | 276-9 |
| 10 | 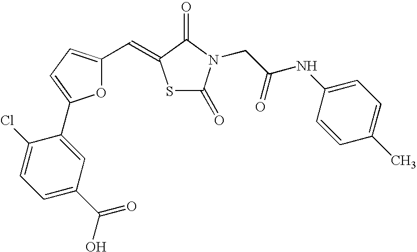 | C24H17ClN2O6S | 276-10 |
| 11 | 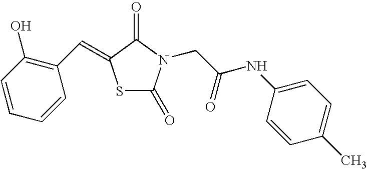 | C19H16N2O4S | 276-11 |
| 12 | 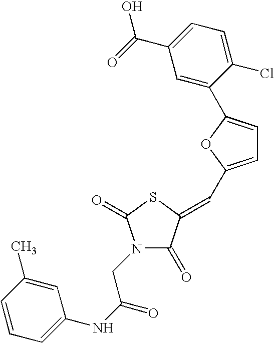 | C24H17ClN2O6S | 276-12 |
| 13 | 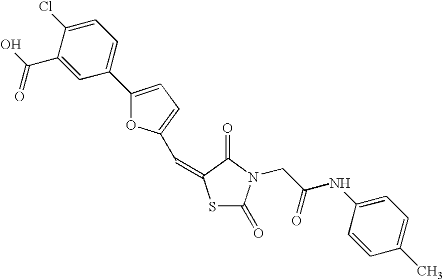 | C24H17ClN2O6S | 276-13 |
| 14 | 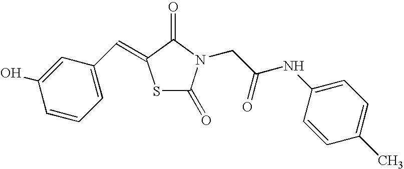 | C19H16N2O4S | 276-14 |
| 15 | 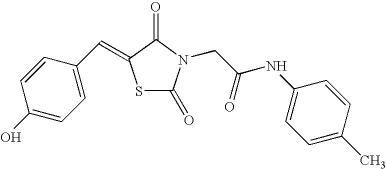 | C19H16N2O4S | 276-15 |
| 16 | 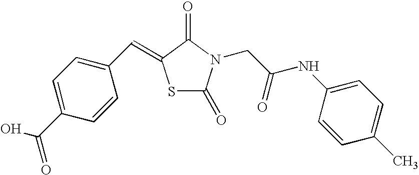 | C20H16N2O5S | 276-16 |
| 17 | 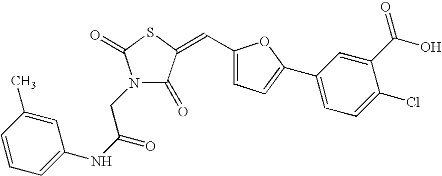 | C24H17ClN2O6S | 276-17 |
| 18 | 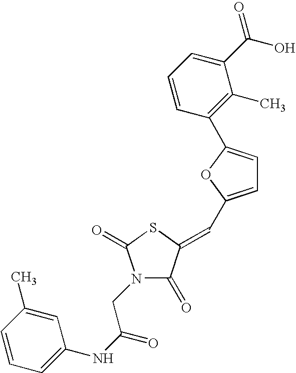 | C25H20N2O6S | 276-18 |
| 19 | 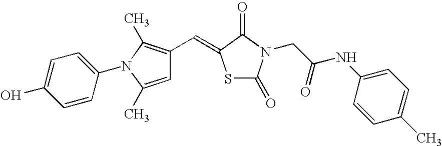 | C25H23N3O4S | 276-19 |
| 20 | 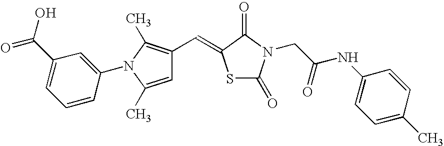 | C26H23N3O5S | 276-20 |
| 21 | 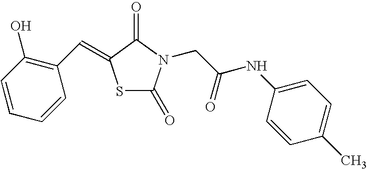 | C19H16N2O4S | 276-21 |
| 22 | 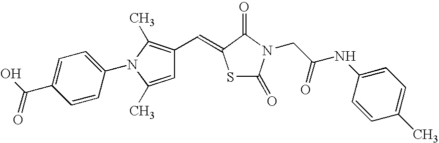 | C26H23N3O5S | 276-22 |
| 23 | 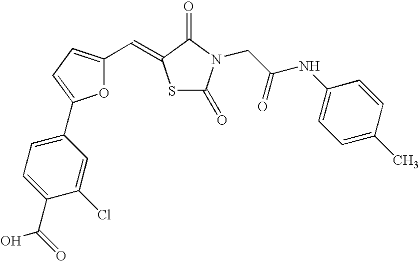 | C24H17ClN2O6S | 276-23 |
| 24 | 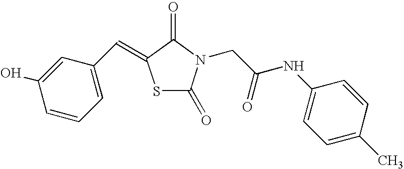 | C19H16N2O4S | 276-24 |
| 25 | 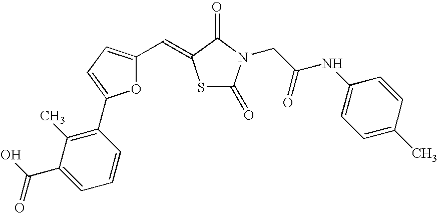 | C25H20N2O6S | 276-25 |
| 26 | 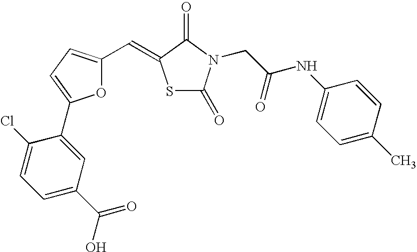 | C24H17ClN2O6S | 276-26 |
| 27 | 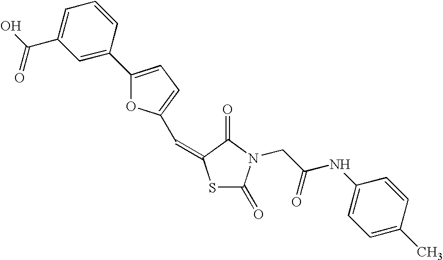 | C24H18N2O6S | 276-27 |
| 28 | 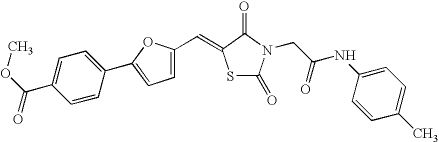 | C25H20N2O6S | 276-28 |
| 29 | 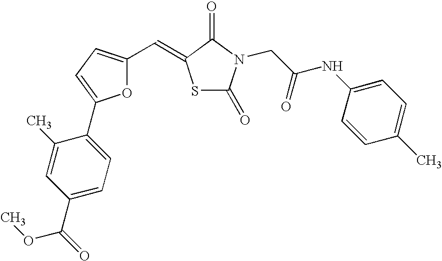 | C26H22N2O6S | 276-29 |

Table 3

| | | | |
|---|---|---|---|
| 1 | 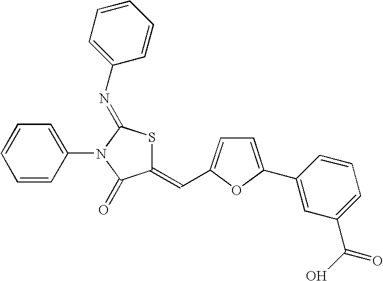 | C27H18N2O4S | 73-1 |
| 2 | 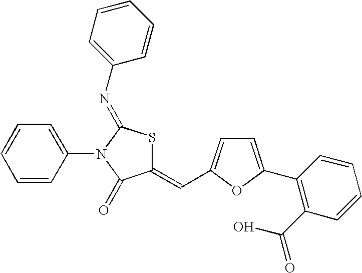 | C27H18N2O4S | 73-2 |
| 3 | 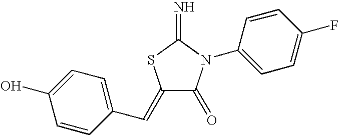 | C16H11FN2O2S | 73-3 |
| 4 | 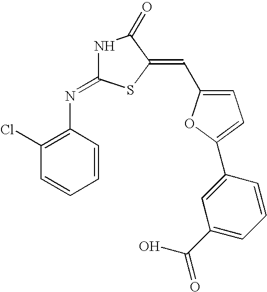 | C21H13ClN2O4S | 73-4 |
| 5 | 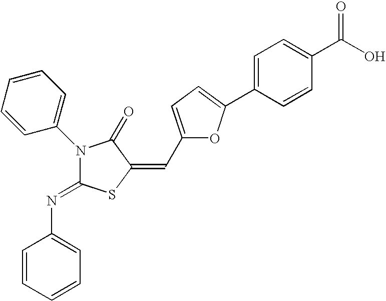 | C27H18N2O4S | 73-5 |
| 6 | 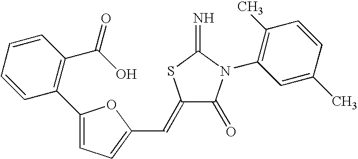 | C23H18N2O4S | 73-6 |
| 7 | 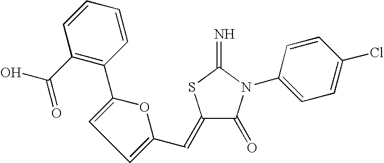 | C21H13ClN2O4S | 73-7 |
| 8 | 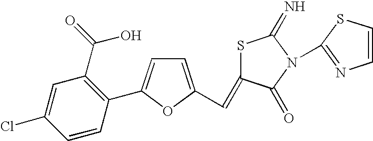 | C18H10ClN3O4S2 | 73-8 |
| 9 | 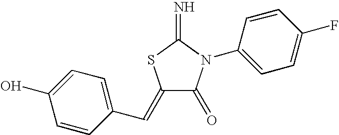 | C16H11FN2O2S | 73-9 |
| 10 | 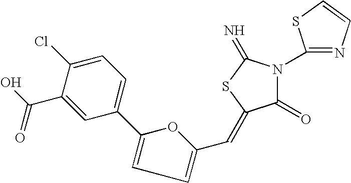 | C18H10ClN3O4S2 | 73-10 |
| 11 | 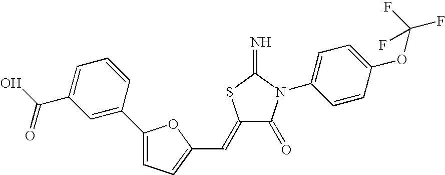 | C22H13F3N2O5S | 73-11 |
| 12 | 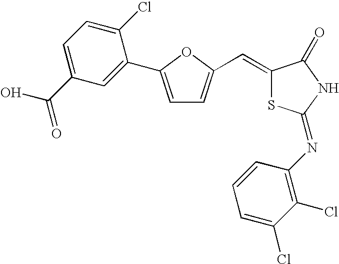 | C21H11Cl3N2O4S | 73-12 |
| 13 | 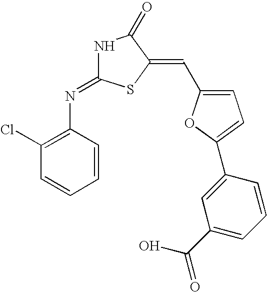 | C21H13ClN2O4S | 73-13 |
| 14 | 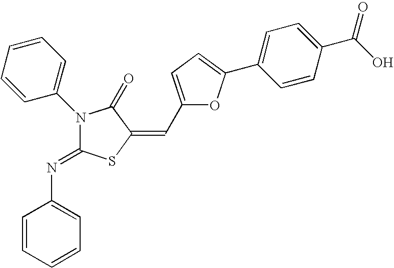 | C27H18N2O4S | 73-14 |
| 15 | 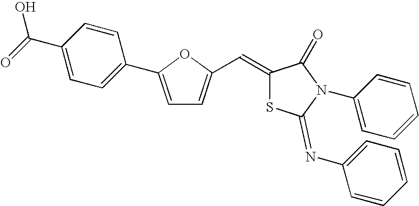 | C27H18N2O4S | 73-15 |
| 16 | 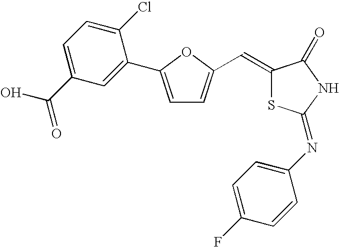 | C21H12ClFN2O4S | 73-16 |
| 17 | 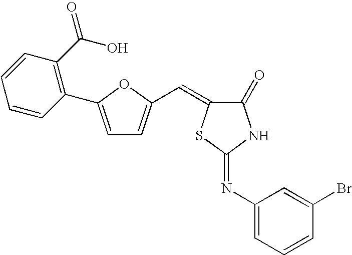 | C21H13BrN2O4S | 73-17 |
| 18 | 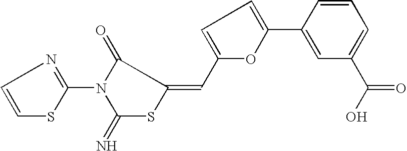 | C18H11N3O4S2 | 73-18 |
| 19 | 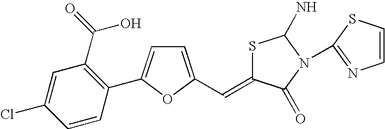 | C18H10ClN3O4S2 | 73-19 |
| 20 | 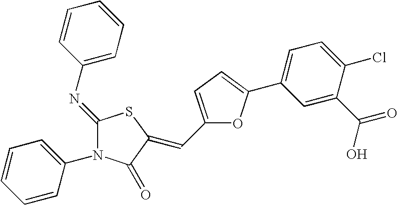 | C27H17ClN2O4S | 73-20 |
| 21 | 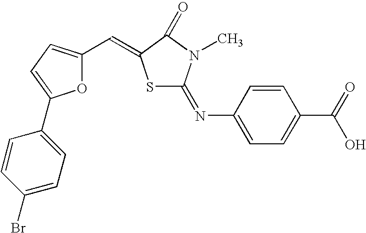 | C22H15BrN2O4S | 73-21 |
| 22 | 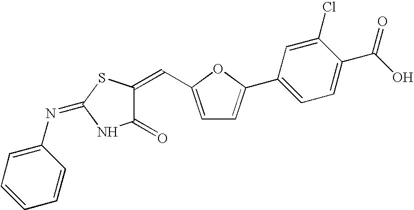 | C21H13ClN2O4S | 73-22 |
| 23 | 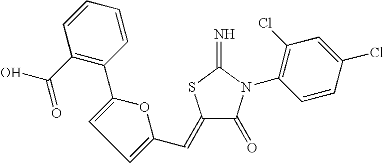 | C21H12Cl2N2O4S | 73-23 |
| 24 | 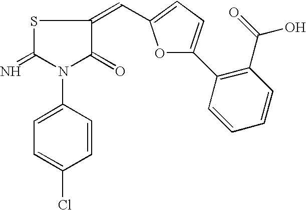 | C21H13ClN2O4S | 73-24 |
| 25 | 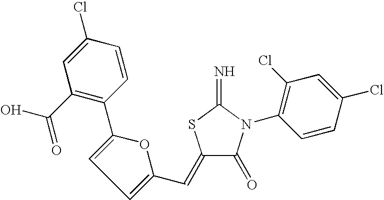 | C21H11Cl3N2O4S | 73-25 |
| 26 | 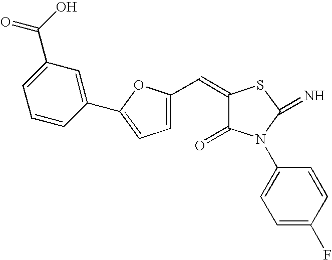 | C21H13FN2O4S | 73-26 |
| 27 | 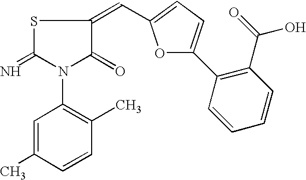 | C23H18N2O4S | 73-27 |
| 28 | 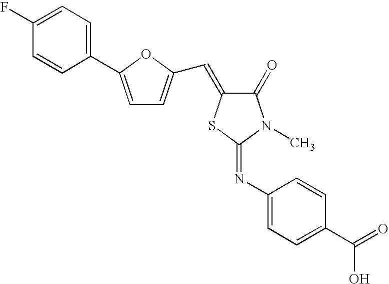 | C22H15FN2O4S | 73-28 |
| 29 | 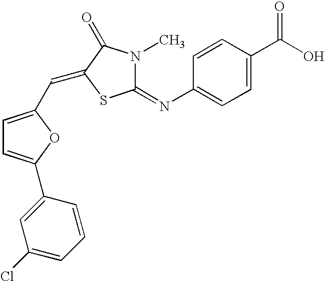 | C22H15ClN2O4S | 73-29 |
| 30 | 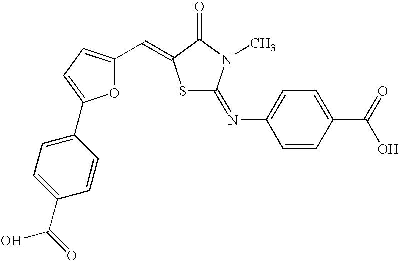 | C23H16N2O6S | 73-30 |
| 31 | 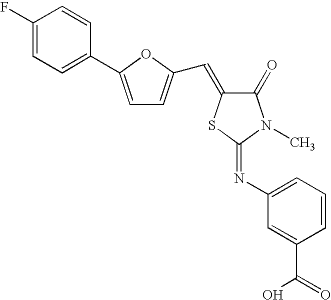 | C22H15FN2O4S | 73-31 |
| 32 | 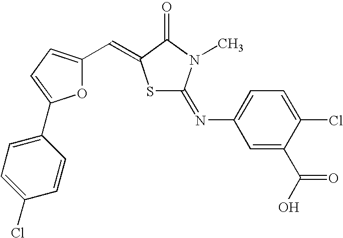 | C22H14Cl2N2O4S | 73-32 |
| 33 | 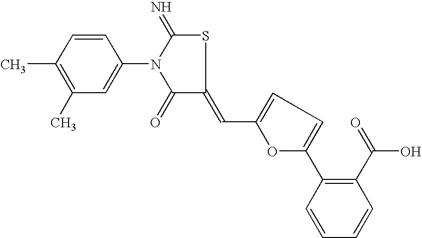 | C23H18N2O4S | 73-33 |

TABLE 2

| | | | |
|---|---|---|---|
| 1 | 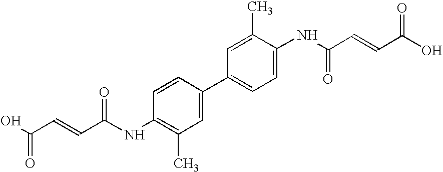 | C22H20N2O6 | 99-1 |
| 2 | 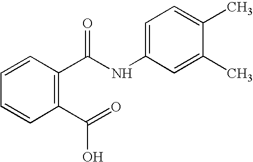 | C16H15NO3 | 99-2 |
| 3 | 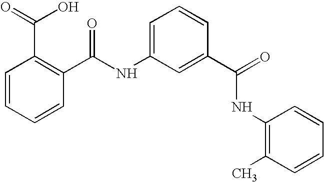 | C22H18N2O4 | 99-3 |
| 4 | 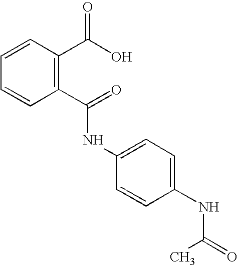 | C16H14N2O4 | 99-4 |
| 5 | 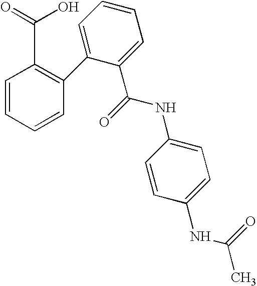 | C22H18N2O4 | 99-5 |
| 6 | 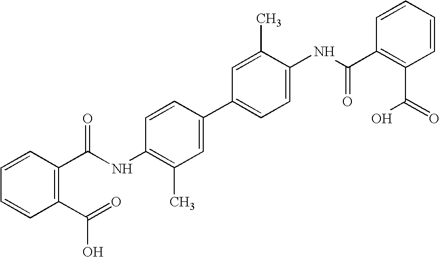 | C30H24N2O6 | 99-6 |
| 7 | 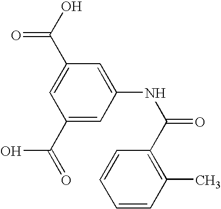 | C16H13NO5 | 99-7 |
| 8 | 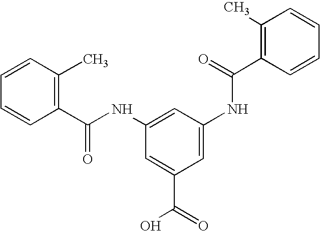 | C23H20N2O4 | 99-8 |
| 9 | 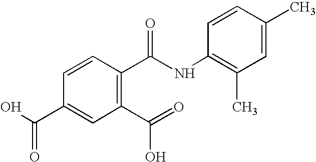 | C17H15NO5 | 99-9 |
| 10 | 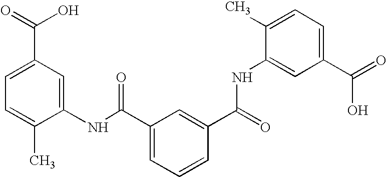 | C24H20N2O6 | 99-10 |
| 11 | 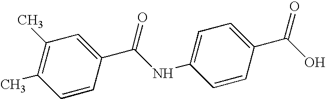 | C16H15NO3 | 99-11 |
| 12 | 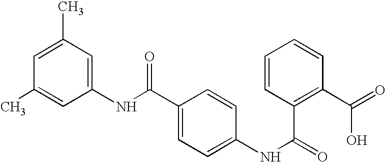 | C23H20N2O4 | 99-12 |
| 13 | 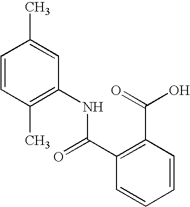 | C16H15NO3 | 99-13 |
| 14 | 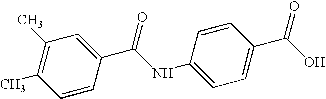 | C16H15NO3 | 99-14 |
| 15 | 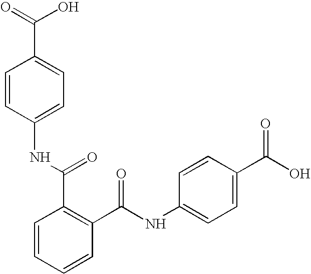 | C22H16N2O6 | 99-15 |
| 16 | 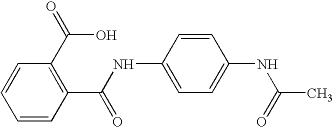 | C16H14N2O4 | 99-16 |
| 17 | 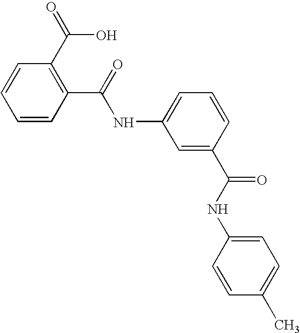 | C22H18N2O4 | 99-17 |
| 18 | 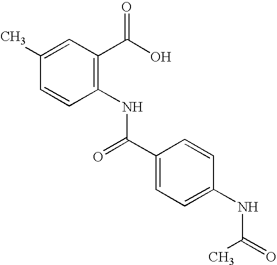 | C17H16N2O4 | 99-18 |
| 19 | 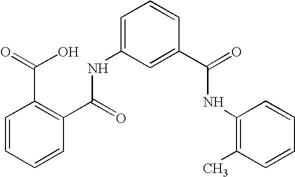 | C22H18N2O4 | 99-19 |
| 20 | 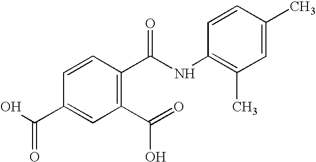 | C17H15NO5 | 99-20 |
| 21 | 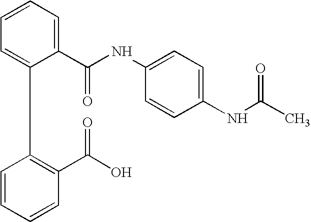 | C22H18N2O4 | 99-21 |
| 22 | 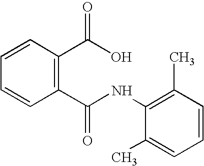 | C16H15NO3 | 99-22 |
| 23 | 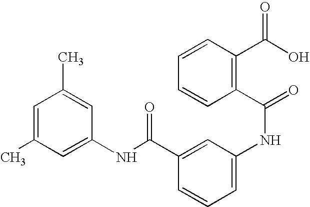 | C23H20N2O4 | 99-23 |
| 24 | 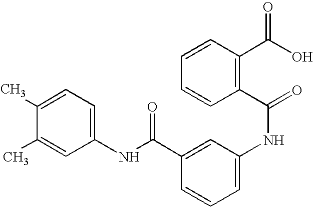 | C23H20N2O4 | 99-24 |
| 25 | 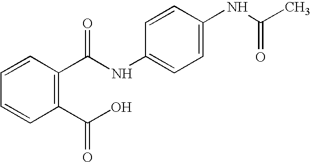 | C16H14N2O4 | 99-25 |
| 26 | 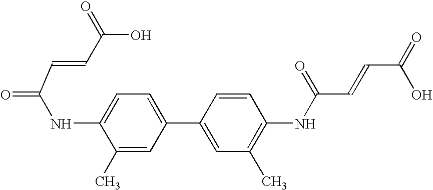 | C22H20N2O6 | 99-26 |
| 27 | 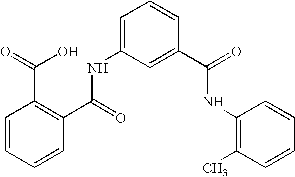 | C22H18N2O4 | 99-27 |
| 28 | 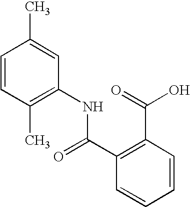 | C16H15NO3 | 99-28 |
| 29 | 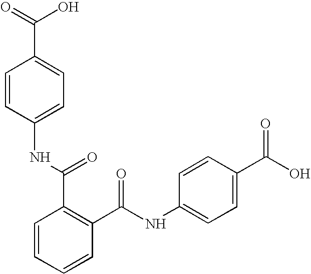 | C22H16N2O6 | 99-29 |
| 30 | 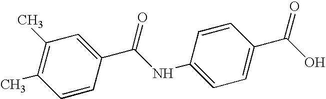 | C16H15NO3 | 99-30 |
| 31 | 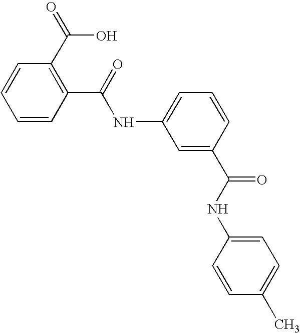 | C22H18N2O4 | 99-31 |
| 32 | 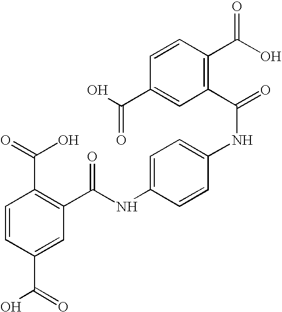 | C24H16N2O10 | 99-32 |
| 33 | 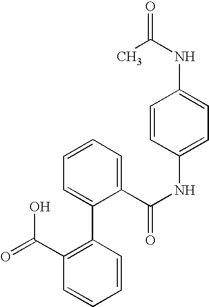 | C22H18N2O4 | 99-33 |
| 34 | 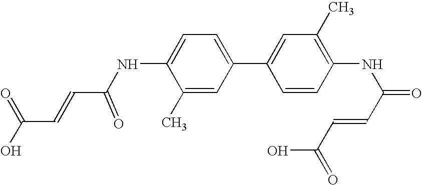 | C22H20N2O6 | 99-34 |
| 35 | 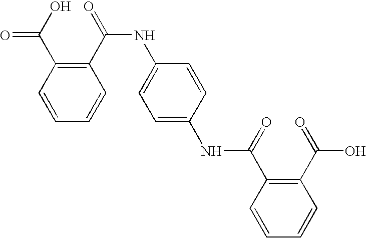 | C22H16N2O6 | 99-35 |
| 36 | 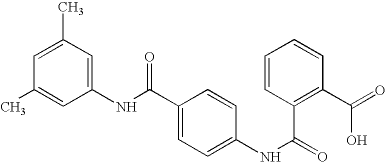 | C23H20N2O4 | 99-36 |
| 37 | 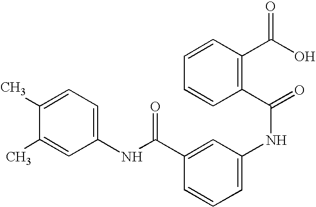 | C23H20N2O4 | 99-37 |

TABLE 4

| | | | |
|---|---|---|---|
| 1 | 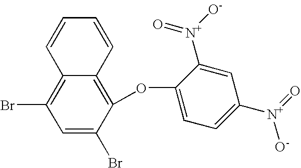 | C15H7Br2N3O5 | 92-1 |
| 2 | 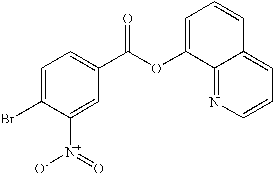 | C16H9BrN2O4 | 92-2 |
| 3 | 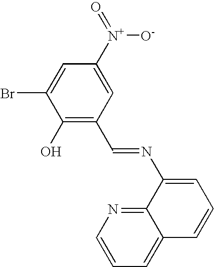 | C16H10BrN3O3 | 92-3 |
| 4 | 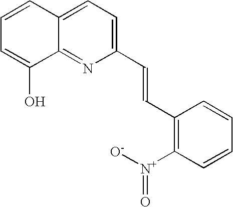 | C17H12N2O3 | 92-4 |
| 5 | 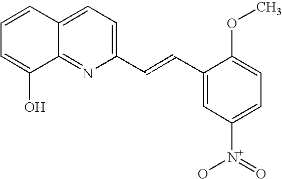 | C18H14N2O4 | 92-5 |
| 6 | 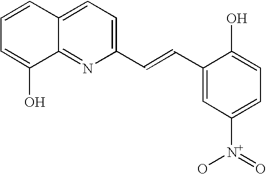 | C17H12N2O4 | 92-6 |
| 7 | 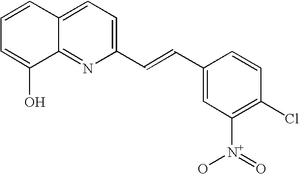 | C17H11ClN2O3 | 92-7 |
| 8 | 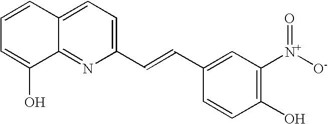 | C17H12N2O4 | 92-8 |
| 9 | 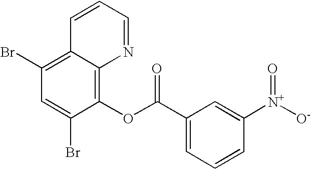 | C16H8Br2N2O4 | 92-9 |
| 10 | 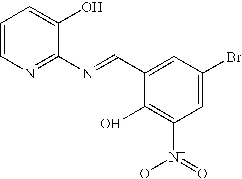 | C12H8BrN3O4 | 92-10 |
| 11 | 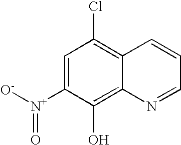 | C9H5ClN2O3 | 92-11 |
| 12 | 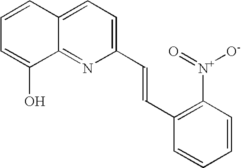 | C17H12N2O3 | 92-12 |
| 13 | 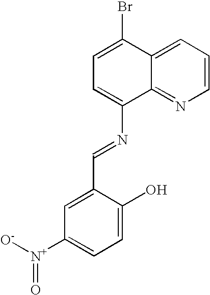 | C16H10BrN3O3 | 92-13 |
| 14 | 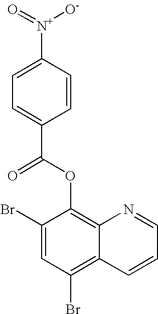 | C16H8Br2N2O4 | 92-14 |
| 15 | 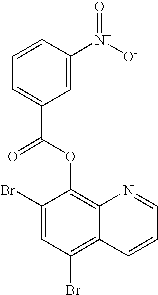 | C16H8Br2N2O4 | 92-15 |
| 16 | 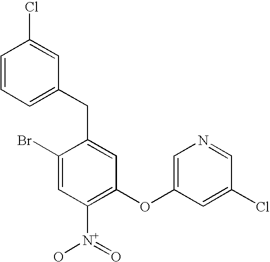 | C16H8BrCl2N3O4 | 92-16 |
| 17 | 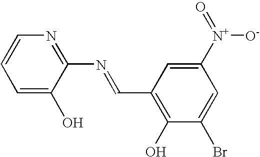 | C12H8BrN3O4 | 92-17 |
| 18 | 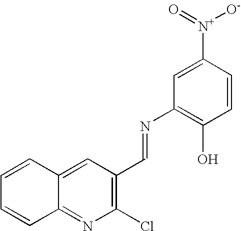 | C16H10ClN3O3 | 92-18 |
| 19 | 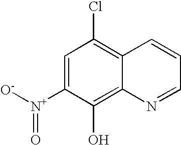 | C9H5ClN2O3 | 92-19 |
| 20 | 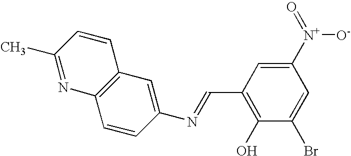 | C17H12BrN3O3 | 92-20 |
| 21 | 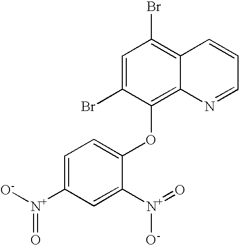 | C15H7Br2N3OS | 92-21 |

TABLE 5

| | | | |
|---|---|---|---|
| 1 | 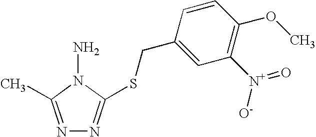 | C11H13N5O3S | 103-1 |
| 2 | 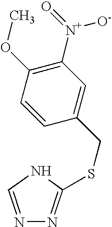 | C10H10N4O3S | 103-2 |
| 3 | 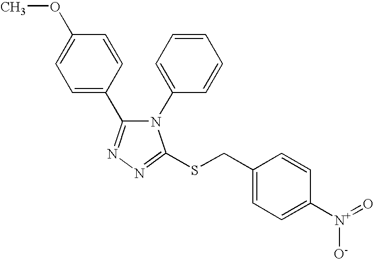 | C22H18N4O3S | 103-3 |
| 4 | 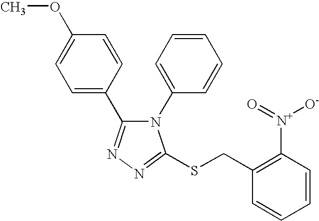 | C22H18N4O3S | 103-4 |
| 5 | 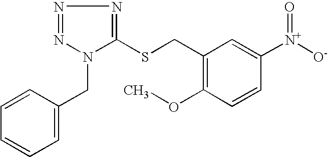 | C16H15N5O3S | 103-5 |
| 6 | 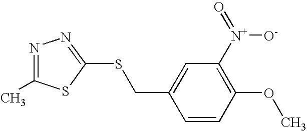 | C11H11N3O3S2 | 103-6 |
| 7 | 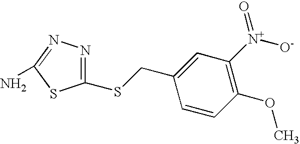 | C10H10N4O3S2 | 103-7 |
| 8 | 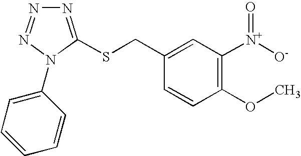 | C15H13N5O3S | 103-8 |
| 9 | 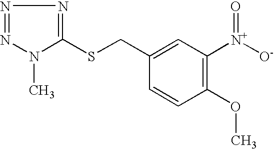 | C10H11N5O3S | 103-9 |
| 10 | 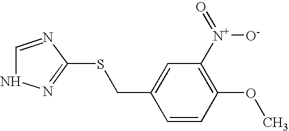 | C10H10N4O3S | 103-10 |
| 11 | 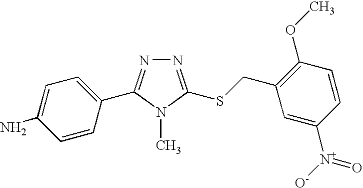 | C17H17N5O3S | 103-11 |
| 12 | 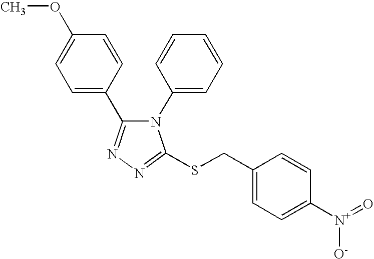 | C22H18N4O3S | 103-12 |
| 13 | 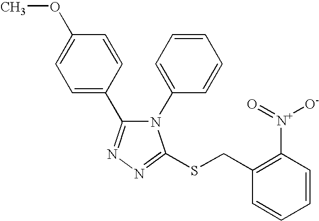 | C22H18N4O3S | 103-13 |
| 14 | 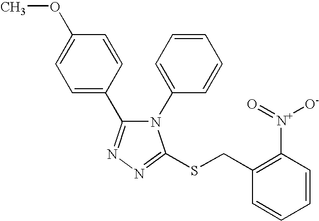 | C22H18N4O3S | 103-14 |
| 15 | 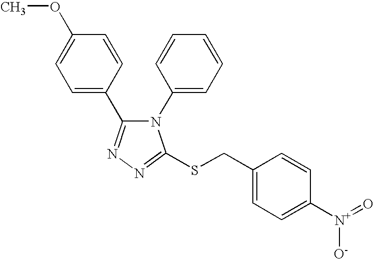 | C22H18N4O3S | 103-15 |
| 16 | 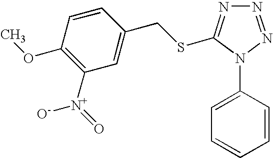 | C15H13N5O3S | 103-16 |
| 17 | 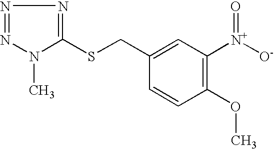 | C10H11N5O3S | 103-17 |
| 18 | 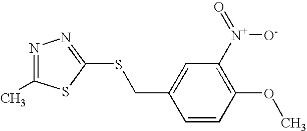 | C11H11N3O3S2 | 103-18 |
| 19 | 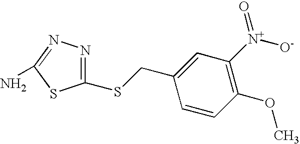 | C10H10N4O3S2 | 103-19 |
| 20 | 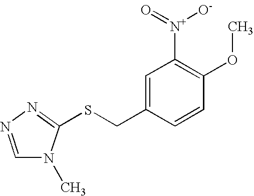 | C11H12N4O3S | 103-20 |

TABLE 6

| | | | |
|---|---|---|---|
| 1 | 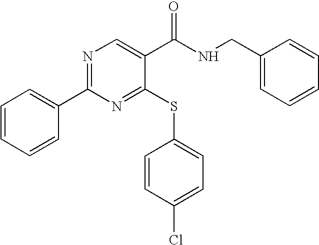 | C24H18ClN3OS | 146-1 |
| 2 | 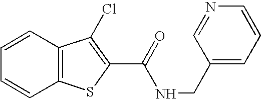 | C15H11ClN2OS | 146-2 |
| 3 | 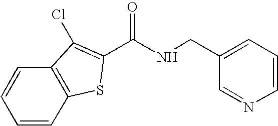 | C15H11ClN2OS | 146-3 |
| 4 | 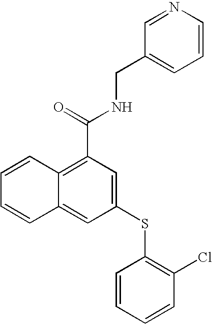 | C22H16ClN3OS | 146-4 |
| 5 | 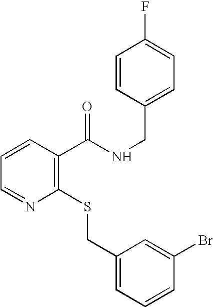 | C20H16BrFN2OS | 146-5 |
| 6 | 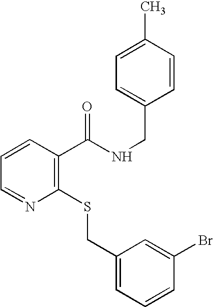 | C21H19BrN2OS | 146-6 |
| 7 | 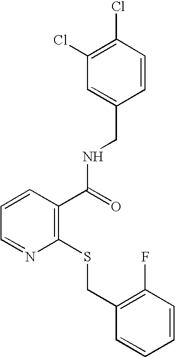 | C20H15Cl2FN2OS | 146-7 |
| 8 | 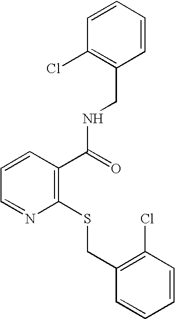 | C20H16C12N2OS | 146-8 |
| 9 | 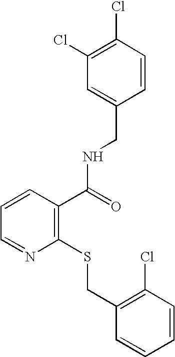 | C20H15Cl3N2OS | 146-9 |
| 10 | 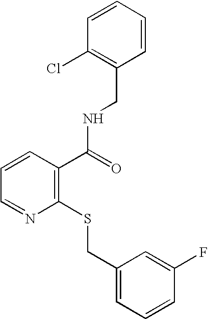 | C20H16ClFN2OS | 146-10 |
| 11 | 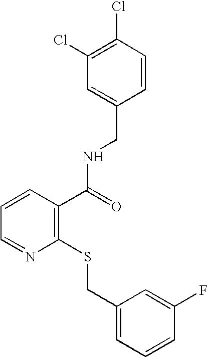 | C20H15Cl2FN2OS | 146-11 |
| 12 | 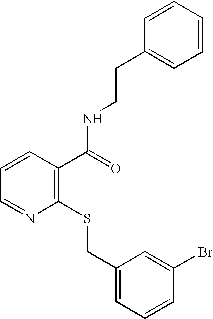 | C21H19BrN2OS | 146-12 |
| 13 | 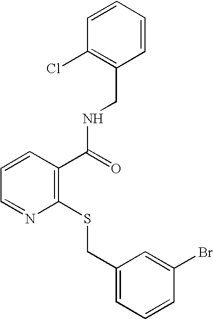 | C20H16BrClN2OS | 146-13 |
| 14 | 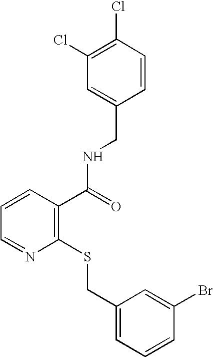 | C20H15BrCl2N2OS | 146-14 |
| 15 | 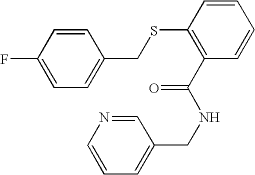 | C20H17FN2OS | 146-15 |
| 16 | 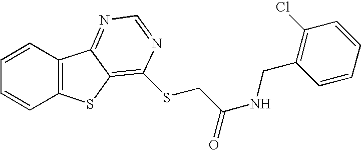 | C19H14ClN3OS2 | 146-16 |
| 17 | 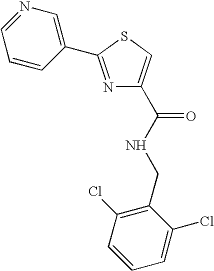 | C16H11Cl2N3OS | 146-17 |
| 18 | 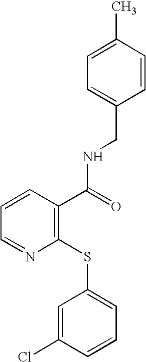 | C20H17ClN2OS | 146-18 |
| 19 | 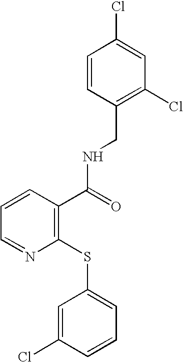 | C19H13Cl3N2OS | 146-19 |
| 20 | 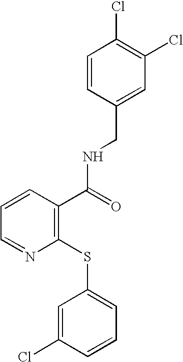 | C19H13Cl3N2OS | 146-20 |
| 21 | 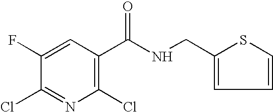 | C11H7Cl2FN2OS | 146-21 |
| 22 | 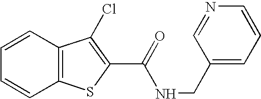 | C15H11ClN2OS | 146-22 |

TABLE 7

| | | | |
|---|---|---|---|
| 1 | 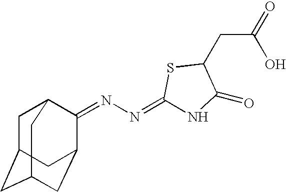 | C15H19N3O3S | 245-1 |
| 2 | 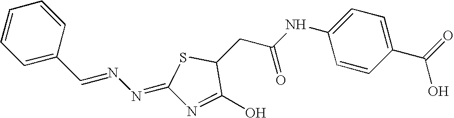 | C19H16N4O4S | 245-2 |
| 3 | 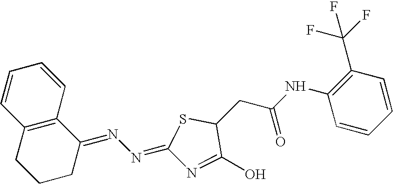 | C22H19F3N4O2S | 245-3 |
| 4 | 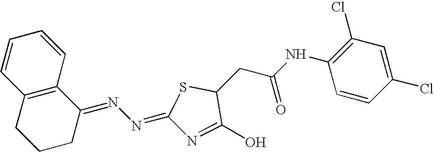 | C21H18Cl2N4O2S | 245-4 |
| 5 | 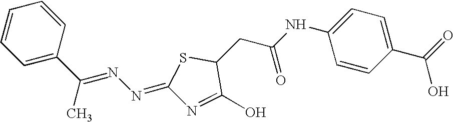 | C20H18N4O4S | 245-5 |
| 6 | 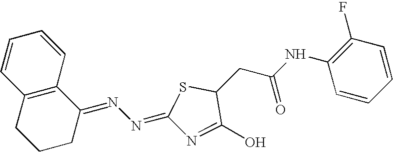 | C21H19FN4O2S | 245-6 |
| 7 | 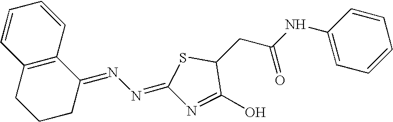 | C21H20N4O2S | 245-7 |
| 8 | 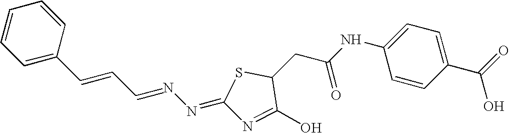 | C21H18N4O4S | 245-8 |
| 9 | 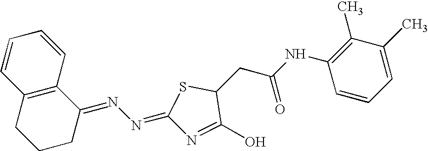 | C23H24N4O2S | 245-9 |
| 10 | 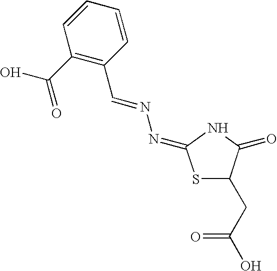 | C13H11N3O5S | 245-10 |
| 11 | 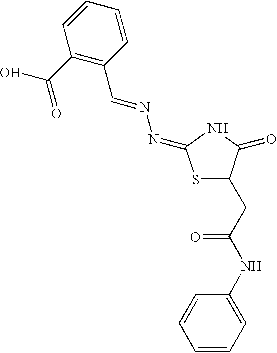 | C19H16N4O4S | 245-11 |
| 12 | 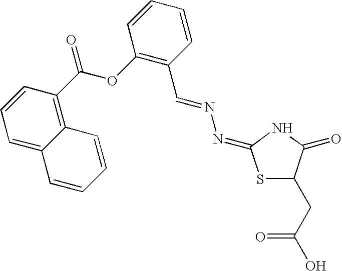 | C23H17N3O5S | 245-12 |
| 13 | 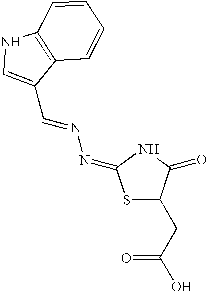 | C14H12N4O3S | 245-13 |
| 14 | 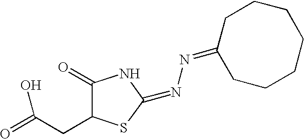 | C13H19N3O3S | 245-14 |
| 15 | 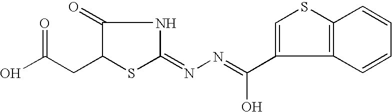 | C15H13N3O3S2 | 245-15 |
| 16 | 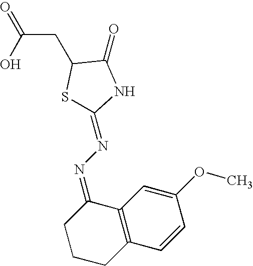 | C16H17N3O4S | 245-16 |
| 17 | 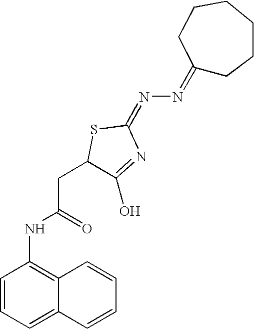 | C22H24N4O2S | 245-17 |
| 18 | 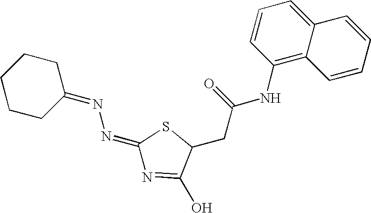 | C21H22N4O2S | 245-18 |
| 19 | 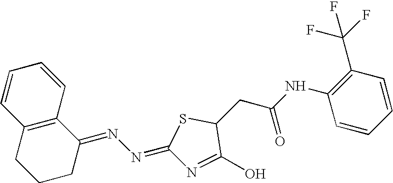 | C22H19F3N4O2S | 245-19 |
| 20 | 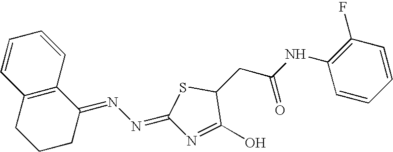 | C21H19FN4O2S | 245-20 |
| 21 | 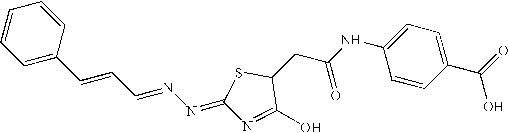 | C21H18N4O4S | 245-21 |
| 22 | 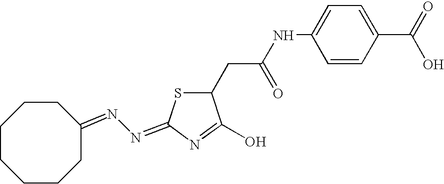 | C20H24N4O4S | 245-22 |
| 23 | 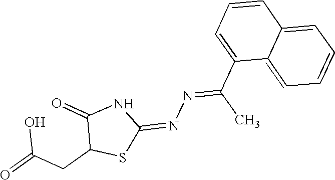 | C17H15N3O3S | 245-23 |
| 24 | 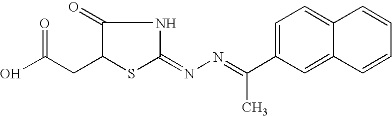 | C17H15N3O3S | 245-24 |
| 25 | 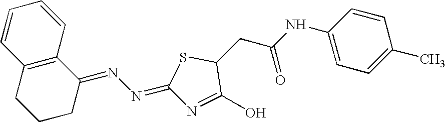 | C22H22N4O2S | 245-25 |
| 26 | 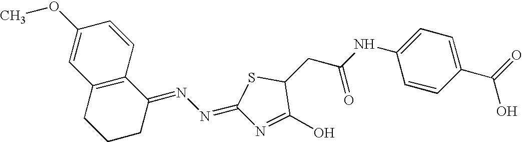 | C23H22N4O5S | 245-26 |

TABLE 8

| | | | |
|---|---|---|---|
| 1 | 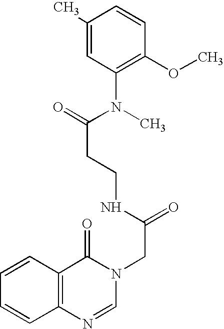 | C22H24N4O4 | 139-1 |
| 2 | 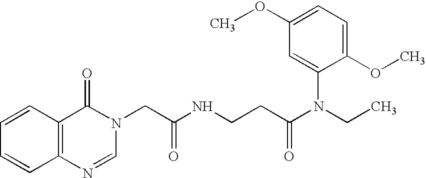 | C23H26N4O5 | 139-2 |
| 3 | 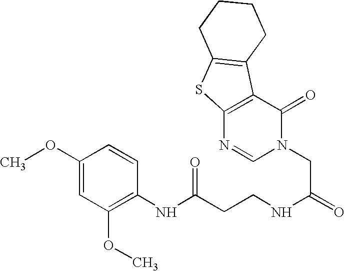 | C23H26N4O5S | 139-3 |
| 4 | 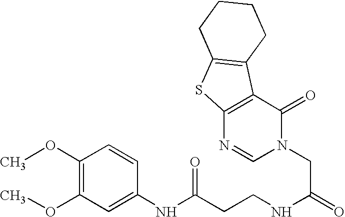 | C23H26N4O5S | 139-4 |
| 5 | 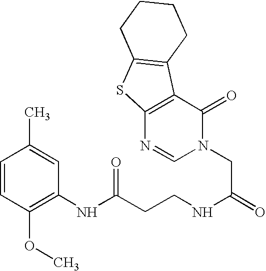 | C23H26N4O4S | 139-5 |
| 6 | 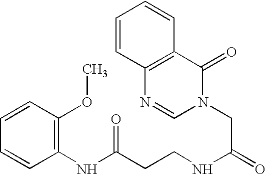 | C20H20N4O4 | 139-6 |
| 7 | 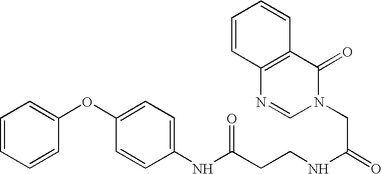 | C25H22N4O4 | 139-7 |
| 8 | 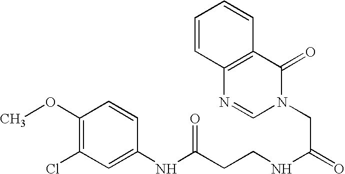 | C20H19ClN4O4 | 139-8 |
| 9 | 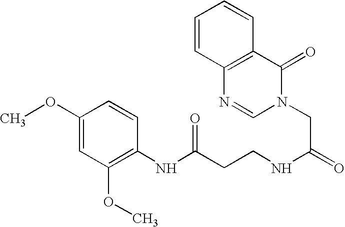 | C21H22N4O5 | 139-9 |
| 10 | 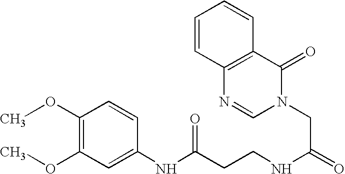 | C21H22N4O5 | 139-10 |
| 11 | 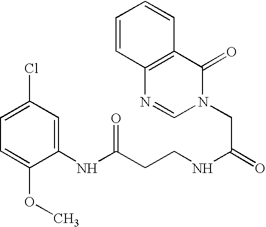 | C20H19ClN4O4 | 139-11 |
| 12 | 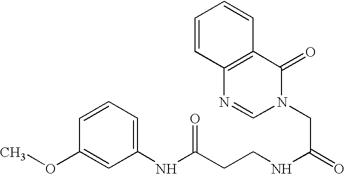 | C20H20N4O4 | 139-12 |
| 13 | 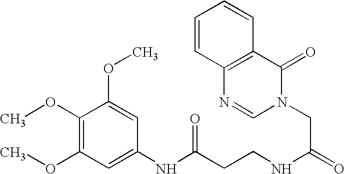 | C22H24N4O6 | 139-13 |
| 14 | 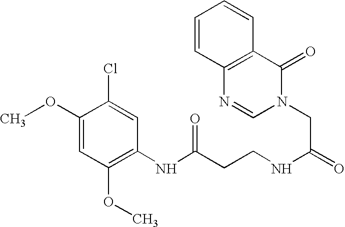 | C21H21ClN4O5 | 139-14 |
| 15 | 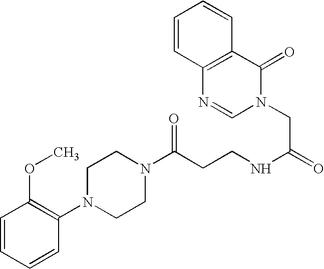 | C24H27N5O4 | 139-15 |
| 16 | 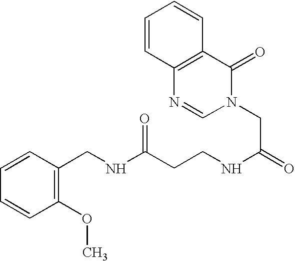 | C21H22N4O4 | 139-16 |
| 17 | 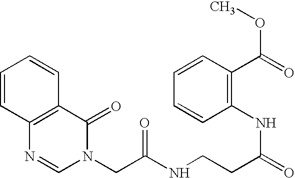 | C21H20N4O5 | 139-17 |
| 18 | 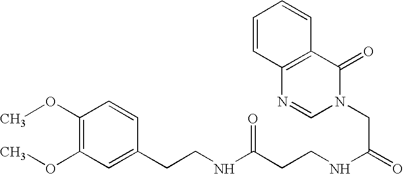 | C23H26N4O5 | 139-18 |
| 19 | 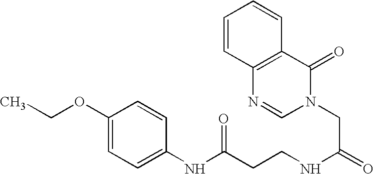 | C21H22N4O4 | 139-19 |
| 20 | 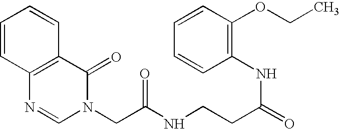 | C21H22N4O4 | 139-20 |
| 21 | 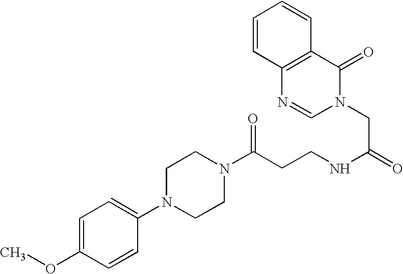 | C24H27NSO4 | 139-21 |
| 22 | 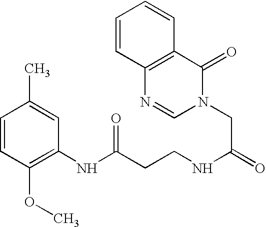 | C21H22N4O4 | 139-22 |
| 23 | 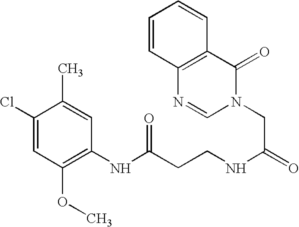 | C21H21ClN4O4 | 139-23 |
| 24 | 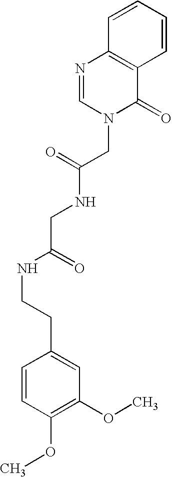 | C22H24N4O5 | 139-24 |
| 25 | 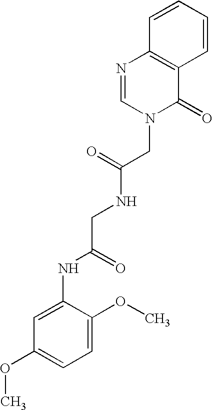 | C20H20N4O5 | 139-25 |
| 26 | 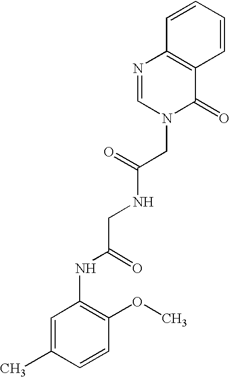 | C20H20N4O4 | 139-26 |

TABLE 9

| | | | |
|---|---|---|---|
| 1 | 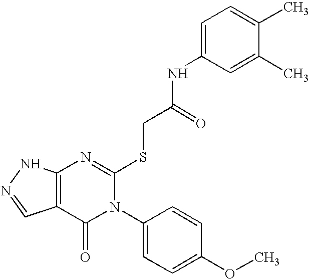 | C22H21N5O3S | 149-1 |
| 2 | 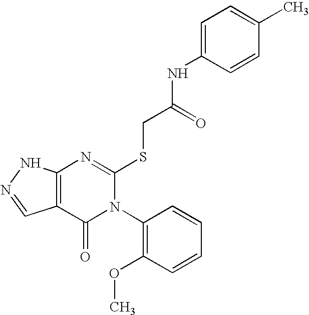 | C21H19N5O3S | 149-2 |
| 3 | 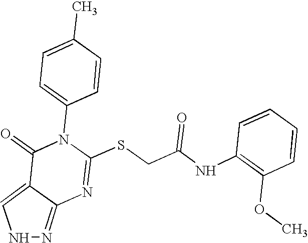 | C21H19N5O3S | 149-3 |
| 4 | 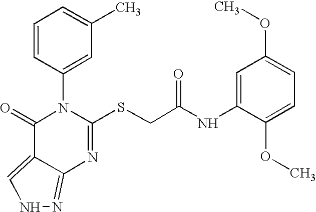 | C22H21N5O4S | 149-4 |
| 5 | 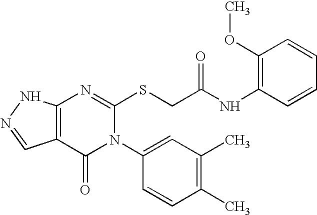 | C22H21N5O3S | 149-5 |
| 6 | 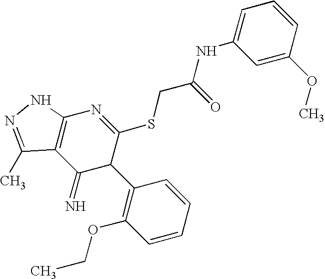 | C23H24N6O3S | 149-6 |
| 7 | 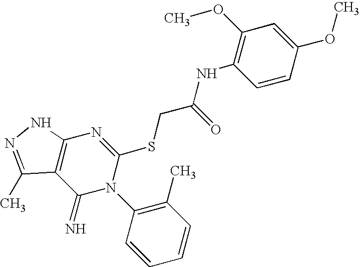 | C23N24N6O3S | 149-7 |
| 8 | 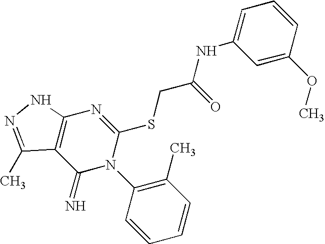 | C22H22N6O2S | 149-8 |
| 9 | 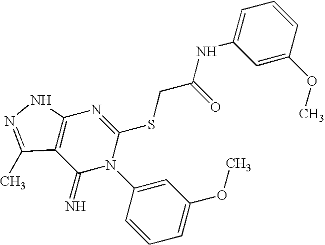 | C22H22N6O3S | 149-9 |
| 10 | 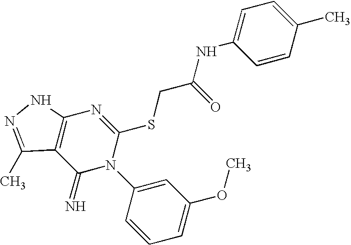 | C22H22N6O2S | 149-10 |
| 11 | 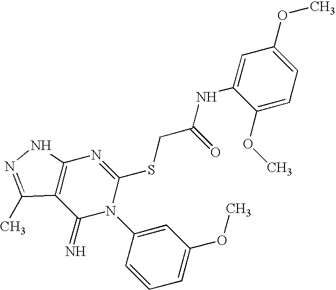 | C23H24N6O4S | 149-11 |
| 12 | 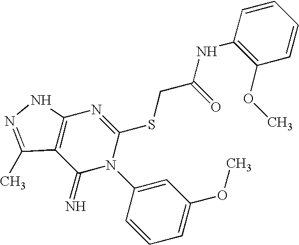 | C22H22N6O3S | 149-12 |
| 13 | 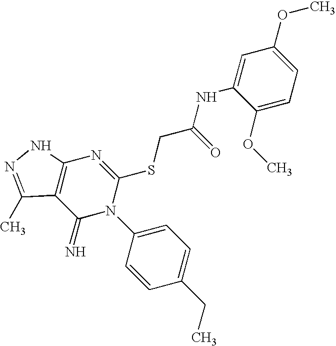 | C24H26N6O35 | 149-13 |
| 14 | 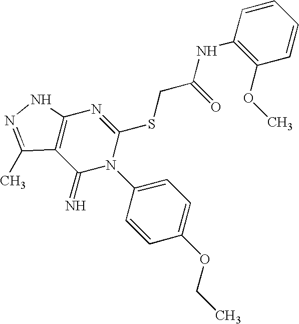 | C23H24N6O3S | 149-14 |
| 15 | 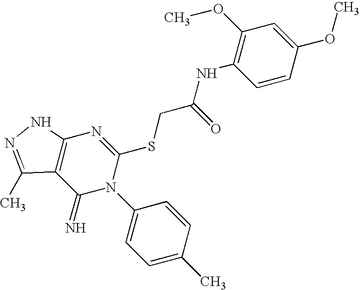 | C23H24N6O3S | 149-15 |
| 16 | 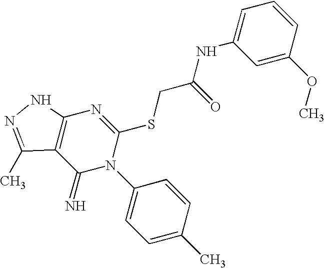 | C22H22N6O2S | 149-16 |
| 17 | 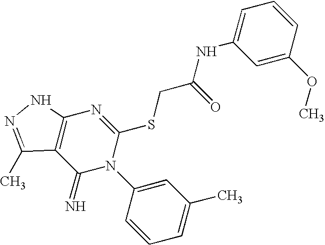 | C22H22N6O2S | 149-17 |
| 18 | 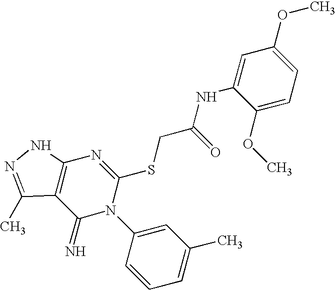 | C23H24N6O3S | 149-18 |
| 19 | 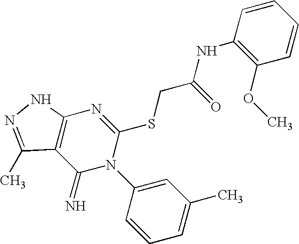 | C22H22N6O2S | 149-19 |
| 20 | 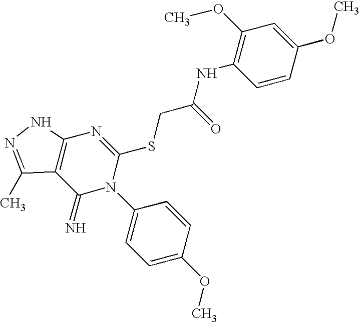 | C23H24N6O4S | 149-20 |
| 21 | 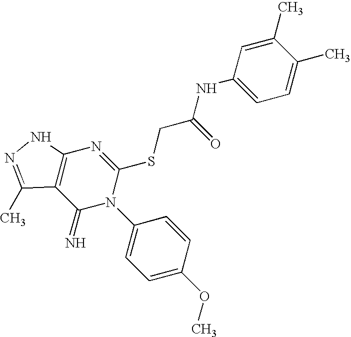 | C23H24N6O2S | 149-21 |
| 22 | 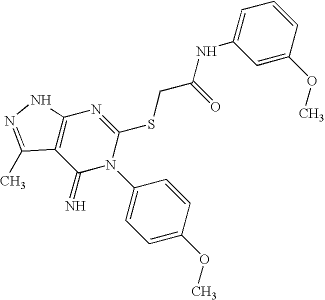 | C22H22N6O3S | 149-22 |
| 23 | 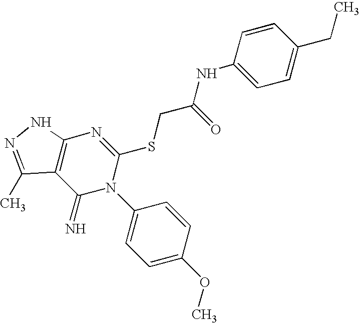 | C23H24N6O2S | 149-23 |
| 24 | 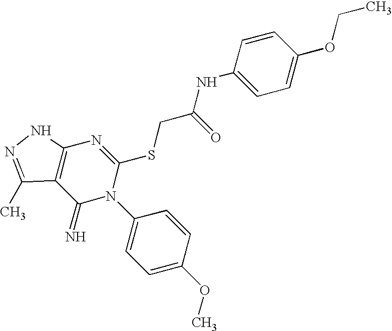 | C23H24N6O3S | 149-24 |
| 25 | 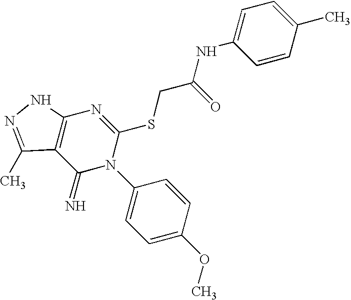 | C22H22N6O2S | 149-25 |
| 26 | 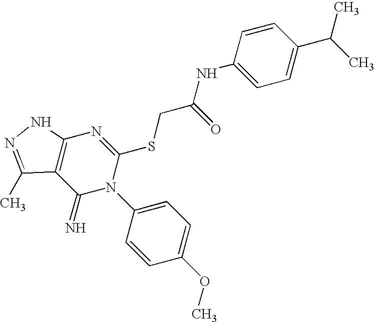 | C24H26N6O2S | 149-26 |
| 27 | 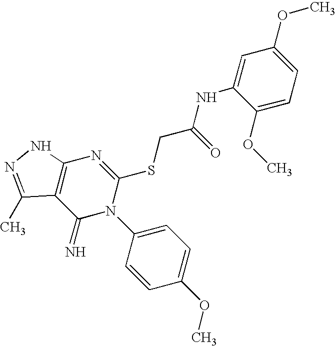 | C23H24N6O4S | 149-27 |
| 28 | 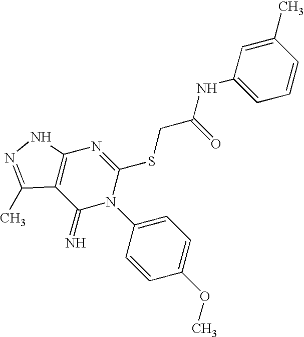 | C22H22N6O2S | 149-28 |
| 29 | 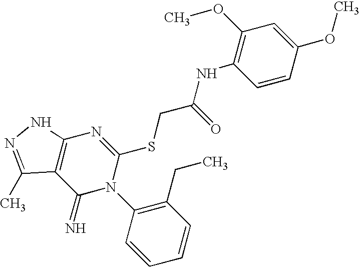 | C24H26N6O3S | 149-29 |
| 30 | 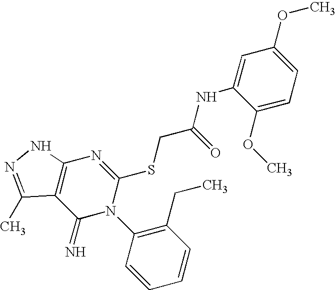 | C24H26N6O3S | 149-30 |

TABLE 10

| | | | |
|---|---|---|---|
| 1 | 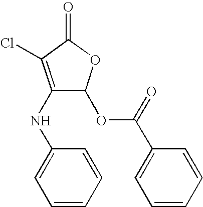 | C17H12C1NO4 | 275-1 |
| 2 | 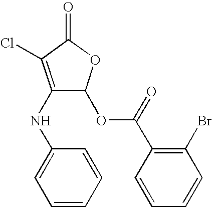 | C17H11BrClNO4 | 275-2 |
| 3 | 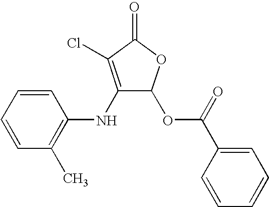 | C18H14ClNO4 | 275-3 |
| 4 | 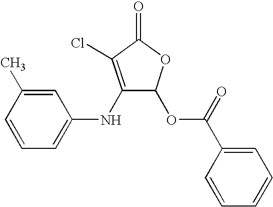 | C18H14ClNO4 | 275-4 |
| 5 | 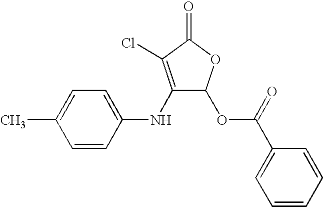 | C18H14ClNO4 | 275-5 |
| 6 | 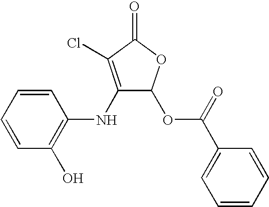 | C17H12ClNO5 | 275-6 |
| 7 | 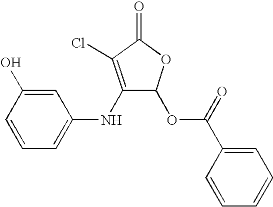 | C17H12ClNO5 | 275-7 |
| 8 | 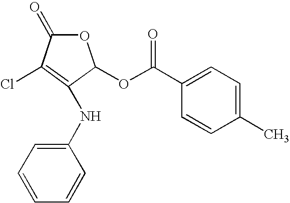 | C18H14ClNO4 | 275-8 |
| 9 | 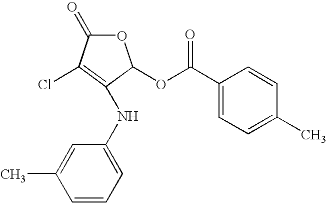 | C19H16ClNO4 | 275-9 |
| 10 | 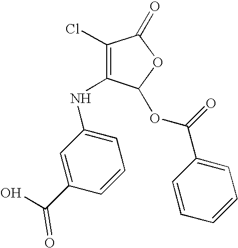 | C18H12ClNO6 | 275-10 |
| 11 | 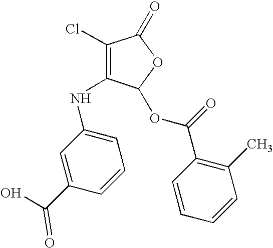 | C19H14ClNO6 | 275-11 |
| 12 | 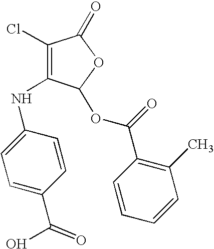 | C19H14ClNO6 | 275-12 |
| 13 | 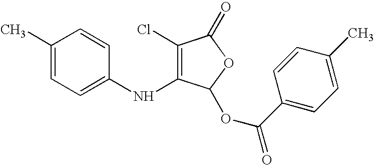 | C19H16ClNO4 | 275-13 |
| 14 | 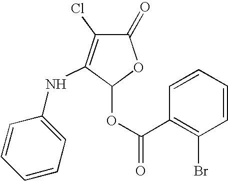 | C17H11BrClNO4 | 275-14 |
| 15 | 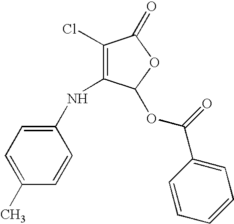 | C18H14ClNO4 | 275-15 |
| 16 | 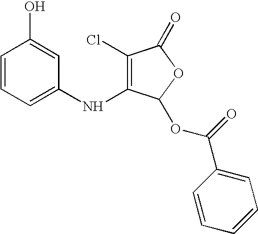 | C17H12ClNO5 | 275-16 |
| 17 | 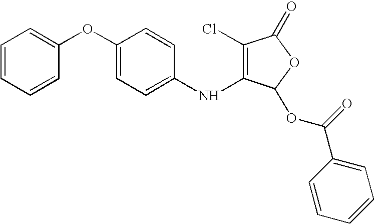 | C23H16ClNO5 | 275-17 |
| 18 | 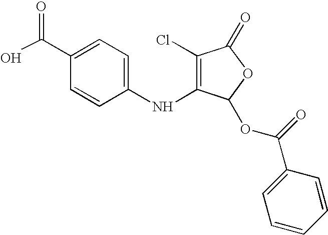 | C18H12ClNO6 | 275-18 |
| 19 | 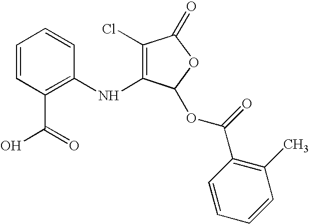 | C19H14ClNO6 | 275-19 |
| 20 | 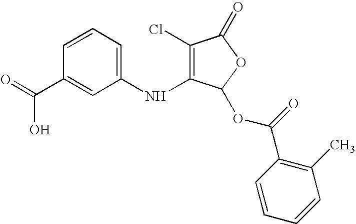 | C19H14ClNO6 | 275-20 |
| 21 | 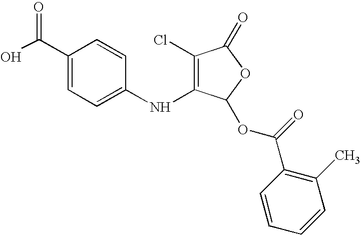 | C19H14ClNO6 | 275-21 |
| 22 | 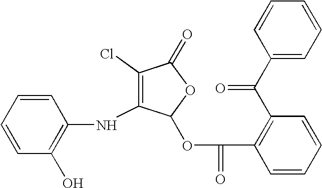 | C24H16ClNO6 | 275-22 |
| 23 | 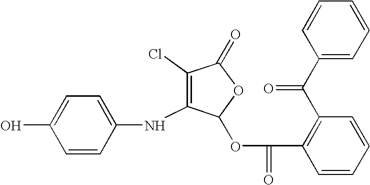 | C24H16ClNO6 | 275-23 |

TABLE 11

| | | | |
|---|---|---|---|
| 1 | 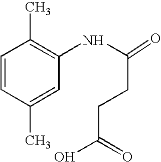 | C12H15NO3 | 162-1 |
| 2 | 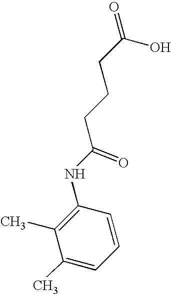 | C13H17NO3 | 162-2 |
| 3 | 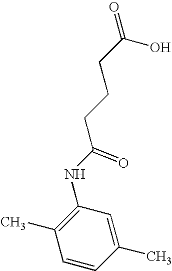 | C13H17NO3 | 162-3 |
| 4 | 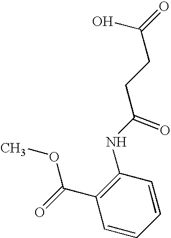 | C12H13NO5 | 162-4 |
| 5 | 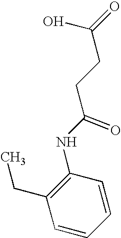 | C12H15NO3 | 162-5 |
| 6 | 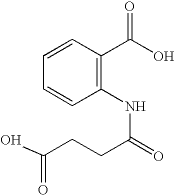 | C11H11NO5 | 162-6 |
| 7 | 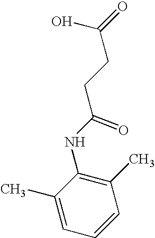 | C12H15NO3 | 162-7 |
| 8 | 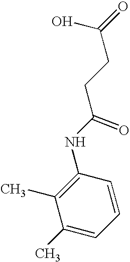 | C12H15NO3 | 162-8 |
| 9 | 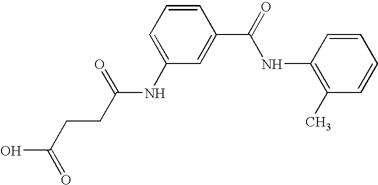 | C18H18N2O4 | 162-9 |
| 10 | 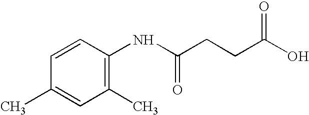 | C12H15NO3 | 162-10 |
| 11 | 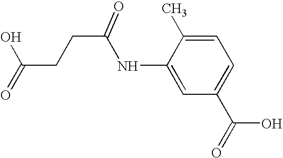 | C12H13NO5 | 162-11 |
| 12 | 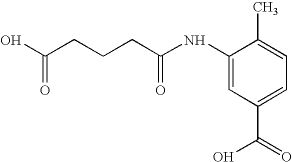 | C13H15NO5 | 162-12 |
| 13 | 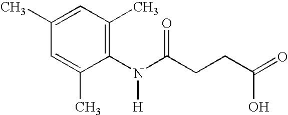 | C13H17NO3 | 162-13 |
| 14 | 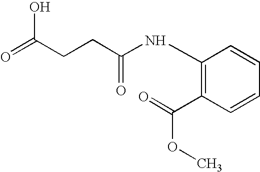 | C12H13NO5 | 162-14 |
| 15 | 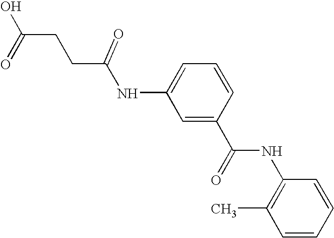 | C18H18N2O4 | 162-15 |
| 16 | 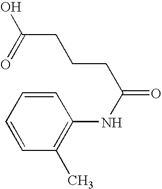 | C12H15NO3 | 162-16 |
| 17 | 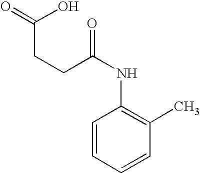 | C11H13NO3 | 162-17 |
| 18 | 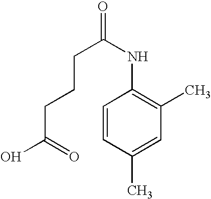 | C13H17NO3 | 162-18 |
| 19 | 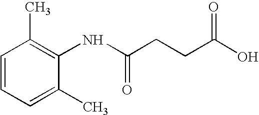 | C12H15NO3 | 162-19 |
| 20 | 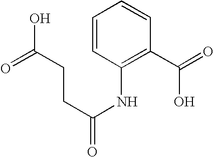 | C11H11NO5 | 162-20 |
| 21 | 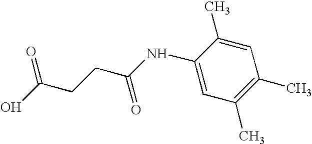 | C13H17NO3 | 162-21 |
| 22 | 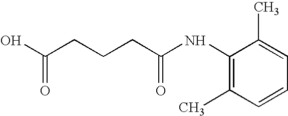 | C13H17NO3 | 162-22 |
| 23 | 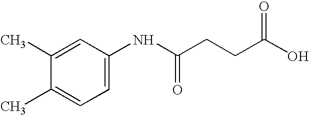 | C12H15NO3 | 162-23 |
| 24 | 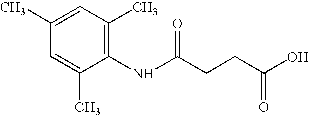 | C13H17NO3 | 162-24 |
| 25 | 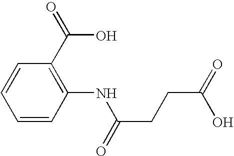 | C11H11NO5 | 162-25 |
| 26 | 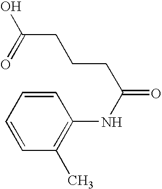 | C12H15NO3 | 162-26 |
| 27 | 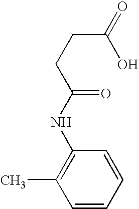 | C11H13NO3 | 162-27 |
| 28 | 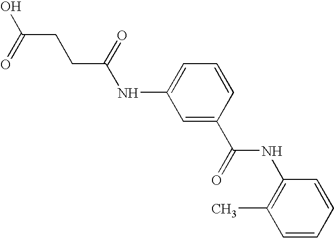 | C18H18N2O4 | 162-28 |
| 29 | 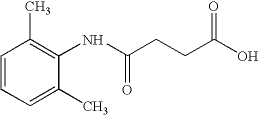 | C12H15NO3 | 162-29 |
| 30 | 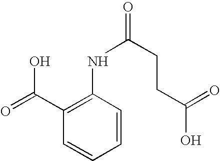 | C11H11NO5 | 162-30 |

TABLE 12

| | | | |
|---|---|---|---|
| 1 | 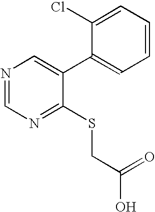 | C12H9ClN2O2S | 262-1 |
| 2 | 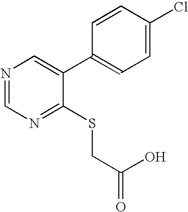 | C12H9ClN2O2S | 262-2 |
| 3 | 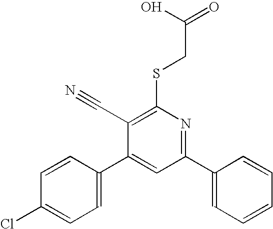 | C20H13ClN2O2S | 262-3 |
| 4 | 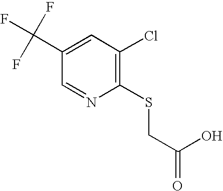 | C8H5ClF3NO2S | 262-4 |
| 5 | 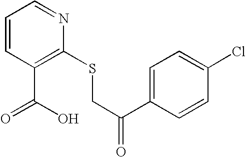 | C14H10ClNO3S | 262-5 |
| 6 | 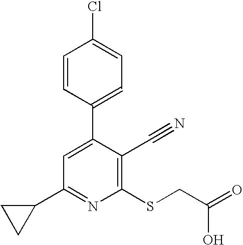 | C17H13ClN2O2S | 262-6 |
| 7 | 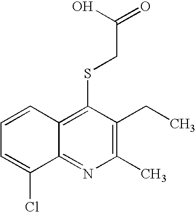 | C14H14ClNO2S | 262-7 |
| 8 | 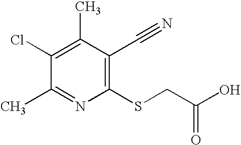 | C10H9ClN2O2S | 262-8 |
| 9 | 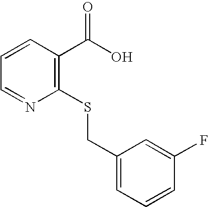 | C13H10FNO2S | 262-9 |
| 10 | 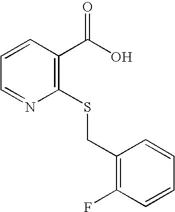 | C13H10FNO2S | 262-10 |
| 11 | 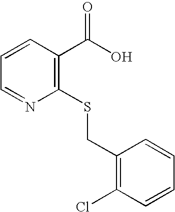 | C13H10ClNO2S | 262-11 |
| 12 | 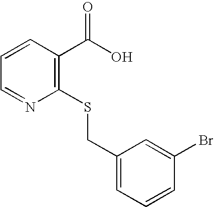 | C13H10BrNO2S | 262-12 |
| 13 | 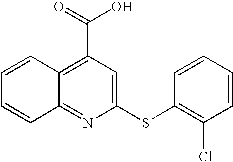 | C16H10ClNO2S | 262-13 |
| 14 | 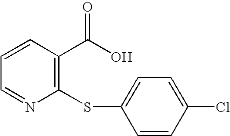 | C12H8ClNO2S | 262-14 |
| 15 | 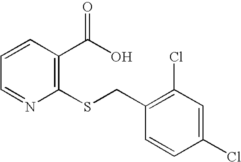 | C13H9Cl2NO2S | 262-15 |
| 16 | 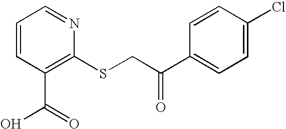 | C14H10ClNO3S | 262-16 |
| 17 | 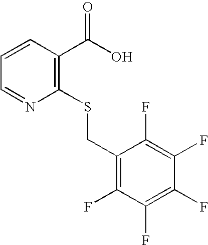 | C13H6F5NO2S | 262-17 |
| 18 | 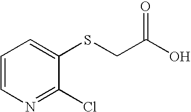 | C7H6ClNO2S | 262-18 |
| 19 | 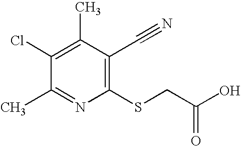 | C10H9ClN2O2S | 262-19 |
| 20 | 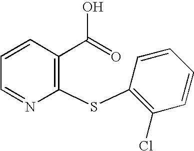 | C12H8ClNO2S | 262-20 |
| 21 | 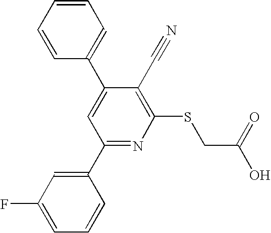 | C20H13FN2O2S | 262-21 |
| 22 | 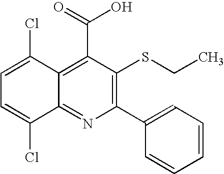 | C18H13C12NO2S | 262-22 |

We claim:

1. A pharmaceutical composition comprising a pharmaceutically acceptable carrier and a compound of formula I, 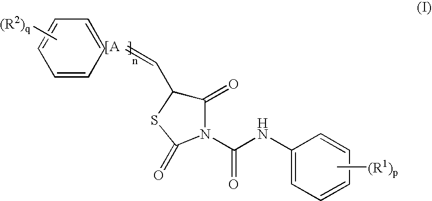
wherein
A is a 5-membered aromatic ring in which optionally a carbon is replaced by a nitrogen or oxygen, and which optionally is substituted in 0, 1 or 2 places with a C₁₋₄ alkyl group, or is a straight chain or branched C₁₋₄ alkenylene group,
n is 0 or 1,
p is 0, 1 or 2,
q is 0, 1 or 2, and
R¹ and R² are, each independently, a halogen atom, a carboxylic acid group, a hydroxyl group, a —C(O)O—C₁₋₄ alkyl group, or a C₁₋₆ alkyl group that is optionally substituted with a hydroxyl group or with a carboxylic acid group,
or a pharmaceutically acceptable salt thereof.

2. A pharmaceutical composition according to claim 1, wherein
R¹ is a C₁₋₄ alkyl group, a halogen atom, or a carboxylic acid group, and
R² is a halogen atom, a carboxylic acid group, a hydroxyl group, a —C(O)O—C₁₋₄ alkyl group, or a C₁₋₄ alkyl group that is optionally substituted with a hydroxyl group.

3. A pharmaceutical composition according to claim 1, wherein
A is 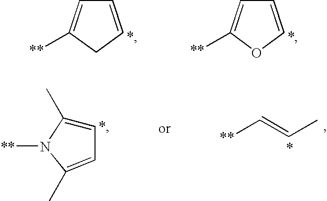
wherein the * denotes the bonding location to the alkenylene group of the compound of formula I, and * * denotes the bonding location to the phenyl ring of the compound of formula I that is adjacent to the group A,
R¹ is CH₃, F, or COOH,
R² is CH₃, Cl, COOH, C(O)OCH₃, OH, or CH₂OH,
n is 0 or 1,
p is 0 or 1, and
q is 0, 1 or 2.

4. A pharmaceutical composition according to claim 1, wherein
R² is a hydroxyl group or carboxylic acid group, and/or
A is

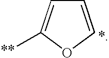

5. A pharmaceutical composition according to claim 1, wherein the compound of formula I is selected from

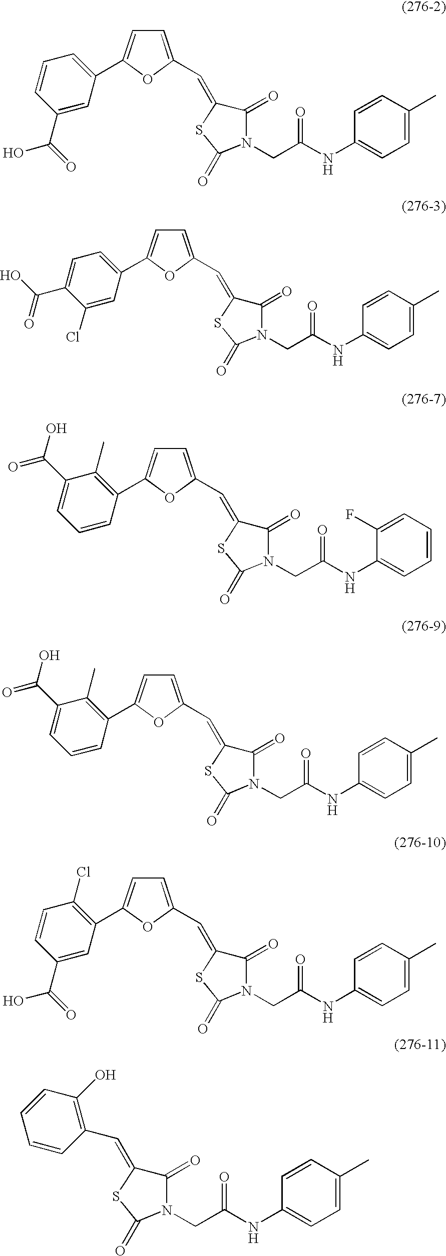

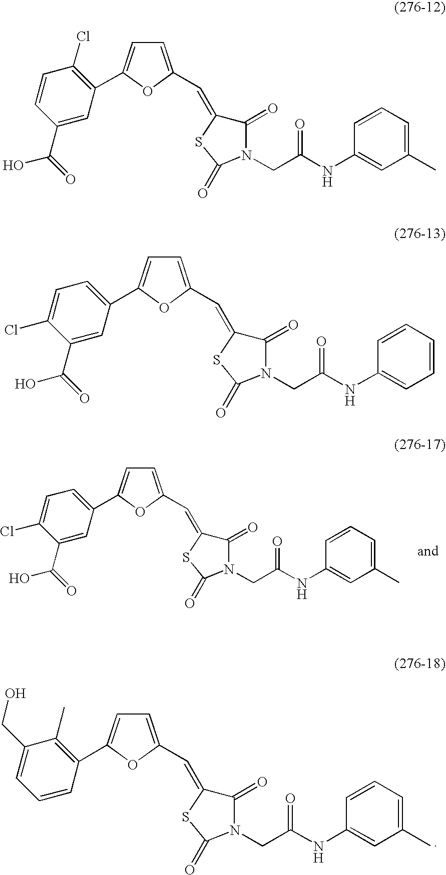

6. A method of achieving an immunomodulatory effect, achieving an antineoplastic effect, or inhibiting hyperproliferative cell growth in a patient in need thereof, comprising administering to said patient an effective amount of a pharmaceutical composition according to claim 1.

7. A method of modulating the binding of a p56^{lck} molecule via an SH2 domain thereof to a corresponding cellular binding protein, or modulating the activity of a p56^{lck} molecule via an SH2 domain thereof, comprising administering a pharmaceutical composition according to claim 1.

8. A method of claim 6, wherein said patient suffers from a transplant rejection.

9. A method of claim 6, wherein said patient suffers from rheumatoid arthritis.

10. A pharmaceutical composition comprising a pharmaceutically acceptable carrier and a compound selected from 276-1 to 276-29, 99-1 to 99-37, 73-1 to 73-33, 92-1 to 92-21, 103-1 to 103-20, 146-1 to 146-22, 245-1 to 245-26, 139-1 to 139-26, 149-1 to 149-30, 275-1 to 275-23, 162-1 to 162-30, and 262-1 to 262-22 and from a pharmaceutically acceptable salt thereof, which compounds are set forth in tables 1 through 12.

11. A method of achieving an immunomodulatory effect, achieving an antineoplastic effect, or inhibiting hyperproliferative cell growth in a patient in need thereof, comprising administering to said patient an effective amount of a pharmaceutical composition according to claim 10.

12. A method of modulating the binding of a p5^{lck} molecule via an SH2 domain thereof to a corresponding cellular binding protein, or modulating the activity of a p56^{lck} molecule via an SH2 domain thereof, comprising administering a pharmaceutical composition according to claim 10.

13. A method of claim 11, wherein immunosuppression is affected.

14. A method of claim 11, wherein said patient suffers from an autoimmune disease.

15. A method of claim 11, wherein said patient suffers from a transplant rejection.

16. A method of claim 11, wherein said patient suffers from rheumatoid arthritis.

17. A method of claim 11, wherein said patient suffers from a neoplasm or a hyperplasia.

18. A method of claim 11, wherein said patient suffers from a benign or malignant tumor.

19. A method of claim 11, wherein said patient suffers from a depressed immune system.

20. A method of claim 11, wherein said patient suffers from leukemia, lymphoma, ovarian cancer and breast cancer.
